(19) United States
(12) Patent Application Publication
(10) Pub. No.: US 2022/0223255 A1
(43) Pub. Date: Jul. 14, 2022

(54) ORTHOPEDIC INTELLIGENCE SYSTEM

(71) Applicant: MedTech S.A., Montpeller (FR)

(72) Inventors: Robert Kraal, Warsaw, IN (US); Michael May, Pittsburgh, PA (US); Ted Spooner, Grand Rapids, MI (US); Mark Brincat, Moreton in Marsh (GB); Matthew Vanderpool, Warsaw, IL (US); Chatherine Leveille, Longueuil (CA)

(21) Appl. No.: 17/574,354

(22) Filed: Jan. 12, 2022

Related U.S. Application Data
(60) Provisional application No. 63/136,962, filed on Jan. 13, 2021.

(51) Int. Cl. G16H 20/40; G16H 40/63; G06N 20/00

(52) U.S. Cl. CPC G16H 20/40; G06N 20/00; G16H 40/63

(57) ABSTRACT

Systems and techniques may be used for providing artificial intelligence regarding orthopedic patients. A technique may include using sensor data generated over a period of time by a patient an input to a machine learning model. The machine learning model may be trained based on labeled sensor data and labeled outcome data. The machine learning model may generate a predicted outcome for the patient. The technique may include output at least one medical intervention recommendation based on the predicted outcome.

### CLAIM OF PRIORITY

This application claims the benefit of priority to U.S. Provisional Application No. 63/136,962 filed Jan. 13, 2021, titled “ORTHOPEDIC INTELLIGENCE SYSTEM,” which is hereby incorporated herein by reference in its entirety.

### BACKGROUND

Orthopedic patient care may require surgical intervention, such as for upper extremities (e.g., a shoulder or elbow), lower extremities (a knee, a hip, etc.) or the like. For example when pain becomes unbearable for a patient, surgery may be recommended. Postoperative care may include immobility of a joint ranging from weeks to months, physical therapy, or occupational therapy. Physical therapy or occupational therapy may be used to help the patient with recovering strength, everyday functioning, and healing. Current techniques involving immobility, physical therapy, or occupational therapy may not monitor or adequately assess range of motion or for pain before or after surgical intervention.

### OVERVIEW

Attempts have been made to make use of advances in computer learning to improve the surgical experience for a patient. For example, US 2019/0019578 to Vaccaro describes a system that purportedly predicts a patient's recovery process. However, the present inventors have recognized that the walking parameters and other patient data used by the system of Vaccaro ignore important data needed by a predictive model.

Furthermore, systems such as the Predict+ system from Exactech, which purports to use machine learning-based software to inform surgeons with potential patient-specific outcomes that may be achieved after shoulder replacement surgery, are based upon a limited training data set and lack access to a continuous, real-time collection of data from multiple patients at varying points of the surgical journey and from multiple data sources.

Moreover, the present inventors have recognized that existing systems utilizing predictive models are an “opaque-box” and fail to provide the physician with any insight as to the relative weight of importance of the input variables associated with the patient. Thus the clinician is left with little opportunity to use her experience to judge the credibility of the prediction.

Furthermore, existing systems consistent of inflexible integrations between existing data sources and do not allow for the patient to contribute her own data to the training data for a predictive model.

Thus, state of the art predictive analytics systems in orthopedic patient care require improvement.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a patient wearing a sensor device in accordance with at least one example of this disclosure.

FIG. 2 illustrates a wearable device in accordance with at least one example of this disclosure.

FIG. 3 illustrates a robotic surgery and feedback system in accordance with at least one example of this disclosure.

FIG. 4 illustrates a machine learning engine for determining feedback in accordance with at least one example of this disclosure.

FIG. 5 illustrates a flowchart showing a technique for providing a predicted outcome of a surgical intervention for a patient in accordance with at least one example of this disclosure.

FIG. 6 illustrates a block diagram of an example machine upon which any one or more of the techniques discussed herein may perform in accordance with at least one example of this disclosure.

FIG. 7 illustrates an overview block diagram for orthopedic interventions in accordance with at least one example of this disclosure.

FIGS. 8A-8E illustrate example user interface timelines for a medical intervention in accordance with at least one example of this disclosure.

FIG. 9 illustrates a surgeon user interface in accordance with at least one example of this disclosure.

FIG. 10 illustrates an overview block diagram for feedback techniques in accordance with at least one example of this disclosure.

FIG. 11 illustrates an example user interface showing a surgical journey in accordance with at least one example of this disclosure.

FIGS. 12A-12C illustrate example user interface information or reports for a surgeon in accordance with at least one example of this disclosure.

FIG. 13 illustrates a flowchart showing a technique for providing a predicted outcome of a surgical intervention for a patient in accordance with at least one example of this disclosure.

FIG. 14 illustrates a flowchart showing a technique for refining a trained model in accordance with at least one example of this disclosure.

FIG. 15 illustrates a flowchart showing a technique for providing a visualization of a medical intervention timeline in accordance with at least one example of this disclosure.

FIG. 16 illustrates a flowchart showing a technique for predicting a patient profile or relevance score in accordance with at least one example of this disclosure.

FIG. 17 illustrates a flowchart showing a technique for providing a predicted outcome of a surgical intervention for a patient in accordance with at least one example of this disclosure.

FIG. 18 illustrates a flowchart showing a technique for providing a platform including medical intervention models in accordance with at least one example of this disclosure.

FIG. 19 illustrates an example user interface showing predictive support analytics information in accordance with at least one example of this disclosure.

### DETAILED DESCRIPTION

Systems and methods described herein may be used for providing a predicted outcome or information related to a predicted outcome of a surgical intervention for a patient. Systems and methods described herein may be used to provide, assess, or augment orthopedic patient care (e.g., upper extremity, hip, knee, etc.). These systems and methods may include pain or range of motion assessment, providing feedback or information to a patient, or augmenting patient care with physical therapy, occupational therapy, warnings, or the like.

The systems and methods described herein may work with, use, or include aspects of any of the following applications: U.S. Publication 2011/0092804, U.S. Publication 2014/0303938, U.S. Publication 2018/0315247, U.S. Publication 2019/0272917, U.S. Publication 2020/0335222, and U.S. application Ser. No. 16/560,793 (U.S. Publication 2021/0065870), each of which is incorporated herein in its entirety.

The Orthopedic Intelligence System collects data from a population of patients. Example populations include patients for a particular surgeon, patients for a particular health system, or patients treated using medical devices from a particular medical device provider. The data collected from the population of patients is used to train one or more predictive and prescriptive analytics models, through supervised learning, unsupervised learning, and other machine learning/artificial intelligence type methods. These models may be developed to provide health care providers (e.g., surgeons) with risk stratification and risk classification for selections of patients, assessments of potential complications for particular patients, recommended interventions (pre-surgical or surgical) for particular patients, recommended surgical approaches and techniques for patients (including intraoperative recommendations based on additional data collected during surgery), chances of post-operative success, recommended post-intervention treatment and therapy plans, risks of failure to comply with prescribed pre- and post-operative therapy regimens (e.g., predicted poor adherence by a particular patient). Importantly, while data from each individual patient (e.g., a “first patient”) is used to further train and evolve the predictive analytics model, the first patient nonetheless enjoys the benefits of the predictive model throughout her episode of care.

Coordination of Data Collection

The data elements described herein as inputs to the Orthopedic Intelligence System are coordinated across a variety of data sources. The Orthopedic Intelligence System may be include information related to the patient. Enrollment in the mymobility system from Zimmer Biomet, for example, allows a medical device manufacturer to receive identification or data of particular patients or begin the process of collecting data from these patients for use with the Orthopedic Intelligence System. Surgical scheduling applications such as the Drive Case Management System (DCMS) by Zimmer Biomet, for example, allow the Orthopedic Intelligence System to coordinate collection of data from a surgical episode of care after a surgical case is logged in DCMS. DCMS or the Orthopedic Intelligence System may be integrated with pre-surgical planning tools, such as the Signature ONE Planner from Zimmer Biomet, for example, to provide preoperative surgical plans (and the associated data) for use in analytics operations. DCMS or the Orthopedic intelligence System may be integrated with computer assisted surgery (CAS) tools to receive data collected intraoperatively during a procedure, for example from various CAS tools such as the eLibra dynamic balancing system, the Sesamoidplasty Optical Navigation system, the ROSA robotics system, or the iAssist inertial navigation system from Zimmer Biomet.

Preoperative Data Collection

A predictive model such as the Orthopedic Intelligence System described herein may be generated using a set of inputs. Collection of preoperative patient data may be used as an input. The preoperative patient data may be used to develop a database of information that may be correlated to final outcomes. This data, which may be called training data, may be used for training machine-learning models configured to recommend protocols or protocol changes based on newly received intra- and post-operative patient data, as described further herein.

Preoperative patient data may be collected through use of mobile devices, such as smart phones or smart wearables (e.g., a smart watch or similar wearable sensor device, such as an ear-worn device). A smart phone may include interactive applications to collect patient engagement and feedback. In some examples, a smart phone or a smart wearable may be used to collect objective measurement data (e.g., range of motion, gait, or step count, among others). An exemplary system for collection of preoperative patient data (and, as discussed herein, post-operative data) is the mymobility platform offered by Zimmer Biomet.

In an example, objective measurement data metrics may be captured, such via a HealthKit system of Apple, Inc., using a wrist-worn wearable sensor, using a smart phone (e.g., via a global positioning satellite (GPS) sensor), an inertial measurement unit, an accelerometer, a gyroscope, a magnetometer, or the like). HealthKit measurement data metrics may include a distance that a user (e.g., a prospective patient) is capable of walking during a walk test (e.g., six-minute walk), an average walking speed of the user, an average length of the step of the user, a percentage of steps in which one foot moves at a different speed than the other when the user is walking (e.g., a Walking Asymmetry Percentage), a percentage of time when both of the user's feet are touching the ground while walking steadily over flat ground (e.g., a Double Support Percentage), the user's speed while climbing a flight of stairs (e.g., a Stair Ascent Speed), the user's speed while descending a flight of stairs (e.g., a Stair Descent Speed), a number of times the user has fallen, the user's heart rate, recorded low and high heart rate events for the user, recorded irregular heart rhythm events, the user's resting heart rate, the user's standard deviation of heartbeat intervals, the user's heart rate while walking, heartbeat or electrocardiogram data for the user, the user's oxygen saturation, the user's body temperature, the user's systolic or diastolic blood pressure, the user's respiratory rate, or the like.

In some examples, personal mobile devices with integrated camera features and orientation data may be used to collect data from the patient with improved accuracy over conventional video-based methods. In some legacy systems, video capture is used in clinical environments for patient pose estimation, which captures range of motion data useful in the orthopedic episode of care. However, patient pose estimation is prone to inaccuracies, requires a visit to the clinician, and is limited to identifying and quantifying specific movements. The systems and techniques described herein use camera data captured by the patient themselves, for example using a personal mobile device with video capture and data transmission capability (e.g., an iPhone). This camera data may be used with corresponding data from a wearable sensor with inertial measurement capabilities (e.g., a smartwatch), to track the patient's daily living activities. A mobile device including three-dimensional sensors, such as an iPhone 12's LiDAR sensor, may be used to further enhance the patient pose estimation process performed by the patient outside a clinical environment. A video recognition model may be trained to categorize motions of a patient from daily living activities based on measured data from the mobile device. In some examples, the video recognition model may not need to use the additional data stream from a wearable. In an example, the wearable sensor data is used to teach (or calibrate) the video recognition model.

The Orthopedic Intelligence System may collect data from other connected devices and applications, such as a sleep ring or wearable, a connected scale (e.g., including current weight, body composition, diet, nutrition, etc.), a food or hydration tracking application (e.g., including user entered food or hydration logs), an exercise tracking application (such as Garmin Connect, Strava, etc.), or the like.

Patients of a relevant population may consult with a surgeon, such as an orthopedic surgeon, to address pain or discomfort in their tissues (e.g., a hip, knee, or shoulder joint). The surgeon may order image data, which may be obtained at a medical imaging facility or a doctor's office. The imaging data may be sent to a manufacturer in an electronic or digital format. In an example, an Orthopedic Intelligence System has access to structural data of the patient's afflicted tissues (e.g., large joint). This structural data may be collected using various imaging technologies such as MRI, CT, X-Ray, fluoroscopy, or 2D X-Ray to 3D (e.g., X-Atlas from Zimmer Biomet, EOS Imaging), Ultrasound, photographs, radiography, point cloud image data, high resolution cameras, hyperspectral cameras, T-ray computed tomography, T-ray diffraction tomography, or the like. Using 2D X-ray data in connection with 3D modeling may leverage the use of lower-cost universal X-ray infrastructure thereby reducing costs.

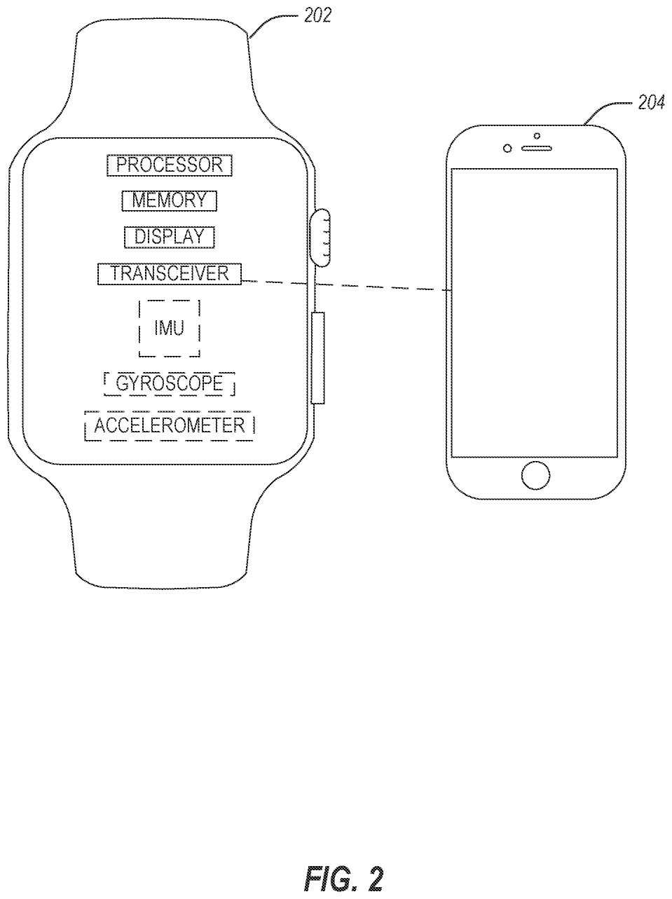

In an example, imaging personnel may access the Orthopedic Intelligence System or DCMS via a network connection and a user device to transmit the imaging data for a patient to a server, where the data may be aggregated with imaging data of other patients. The imaging personnel may access the Orthopedic Intelligence System (or DCMS) via a browser on the user device. The Orthopedic Intelligence System may cause the user device to display a user interface in the form of a web portal, login page, or application, examples of which (and devices for so displaying) are schematically illustrated in FIGS. 2, 3, 8A-8F, 9, and 12A-12C.

The degree and type of degradation of the patient's afflicted joints may be stored by the Orthopedic Intelligence System. This information may be generated based on a human evaluation of the imaging data (e.g., diagnosis by the physician), or based on a trained image recognition system (e.g., machine-learning based) to automatically identify or categorize defects in the patient's hard or soft tissues as captured in the imaging. For example, the image recognition model may recognize medial compartmental osteoarthritis from one or more images of the patient's knee, determine a Walch classification of glenoid morphology from one or more images of an afflicted patient's shoulder, or the like. In an example, the image recognition model may recognize bone marrow lesions from MRI, which have been identified as treatable by early intervention techniques such as Subchondroplasty by Zimmer Biomet.

An ear-worn device may include a sensor to capture data over a period of time. The ear-worn device may include a communication component to send a set of previously captured data from the period of time to a processing device, and receive instructions for outputting audio based on a medical intervention timeline determined by the processing device based on the set of previously captured data and a trained model. The ear-worn device may include a speaker to output audio based on the instructions.

Preoperative data collection may include collection of the patient's desired outcomes or post-operative goals, including desired activities or lifestyle goals of the patient. According to one example, the patient may identify physical activities that they desire to participate in, including those outside of or in addition to daily living. In this regard, some patients may desire a hip, knee, or shoulder joint prosthesis that provides the patient with a range of motion suitable for participating in physical activities such as, by way of example, yoga, downhill skiing, kick-boxing, rowing, etc. These lifestyle goals or activities may be collected through interview of the patient by a care team member, or through self-reporting via a patient engagement application such as mymobility. This desired outcome data may be transmitted electronically via a network to the Orthopedic Intelligence System.

Other preoperative background information from the patient may be collected via interview with the patient, through an application such as mymobility, or through integration with other electronic medical records (EMR or EHR) systems. For example, a patient file of the Orthopedic Intelligence System described herein may include personal and medical information of the patient, such as, for example, weight, height, BMI, gender, age, lifestyle, pertinent medical records, or medical history. The application may make use of Patient Report Outcome Measures (PROMs) that are collected via questionnaire to the patient on a periodic basis preoperatively, such as KOOS, HOOS, WOMAC, Forgotten Joint Score, as described in U.S. Patent Application Publication US20180261316A1 to Zimmer US, Inc., which is incorporated by reference herein in its entirety. As described further herein, the questionnaire data may be collected post operatively and stored in the Orthopedic Intelligence System.

Medical history collected by the Orthopedic Intelligence System may include the patient's (and the patient's family) history of infectious and parasitic diseases, neoplasms, diseases of the blood and blood-forming organs and disorders involving the immune mechanism, endocrine, nutritional and metabolic diseases, mental, behavioral and neurodevelopmental disorders, diseases of the nervous system, diseases of the eye and adnexa, diseases of the ear and mastoid process, diseases of the circulatory system, diseases of the respiratory system, diseases of the digestive system, diseases of the skin and subcutaneous tissue, diseases of the musculoskeletal system and connective tissue, diseases of the genitourinary system, pregnancy, childbirth and the puerperium, conditions originating in the perinatal period, congenital malformations, deformations and chromosomal abnormalities, abnormal clinical and laboratory findings, injuries, poisonings, or the like. The Orthopedic Intelligence System may collect the patient's allergy information.

In addition to or instead of data collected from the patient, relevant data may be collected by the Orthopedic Intelligence System regarding the expertise, experience, and preferences of the patient's surgeon. This information may be collected from the DCMS case management system, or from databases of physician information, for example Healthgrades (physician and hospital reviews), LinkedIn (educational and professional background), or Doximity (physician social media profiles). Exemplary data elements may include types or number of surgeries performed, years of experience, geographical area of practice, preferred computer-assisted technology (navigation, robotics, patient-specific guides, etc.), practice setting, training courses completed, reviews, complaints, surgical approaches, or the like.

The system may leverage surgeon preferences for each individual type of surgical procedure. For example, each surgeon may enter data describing their preferred approach to each individual surgical procedure. In an example, the system captures an individual surgical approach (e.g., preferred implants and preferred techniques) and uses these with a predictive model. In some example, the individual surgical approach may be related to the patient outcome data for determining a specific implant or technique to be used for each individual patient. For example, in a total knee arthroplasty procedure different surgeons may prefer different implants, different implant sizing, different implant positioning (rotation, varus/valgus angles, etc. . . . ), different soft-tissue balancing measurements, different gap balancing, different resection techniques, or other metrics. The predictive engine with the Orthopedic Intelligence System may provide different recommendations to a surgeon that prefers a medial parapatellar approach versus a midvastus or subvastus approach. In another example, the predictive engine may be trained to override a surgeon's preferred approach based on other patient data inputs. In cases where the system provides a recommendation to override a surgical preference, the system may be configured to provide objective data to the surgeon detailing why the predictive engine provided the override recommendation.

Intraoperative Data Collection

One aspect of improving patient outcomes may include correlating intra-operative information collected during a procedure with final patient outcomes. For example, this correlation may include collection of objective intraoperative measures, such as soft tissue tension, implant orientation, etc., to be correlated with postoperative protocol or outcome data. The correlations may be used to guide postoperative care towards successful outcomes or to avoid unsuccessful or less than ideal outcomes.

In an example of a total knee arthroplasty, intra-operative data elements (which may include elements native or as corrected by surgery) may be captured by the Orthopedic Intelligence System from a patient's surgery (and, optionally, data elements recommended by the Orthopedic Intelligence System's predictive model for other patients). These intra-operative data elements may include data captured from an implant family, an implant brand, an instrumentation (e.g., anterior referencing or posterior referencing), a cemented versus press fit fixation, a choice of cut block, a surgical direction (e.g., direct anterior or anteromedial), a technology used (e.g., robotics, patient specific instrumentation, optical navigation, inertial navigation), a tibial or femoral valgus or varus angle relative to a mechanical axis or an anatomical axis, a hip-knee-angle, a distal femoral resection angle, a proximal tibial resection angle, a distal femur resection thickness (lateral and medial), a proximal tibia resection thickness (e.g., medial or lateral, or optionally with the reference point used in the thickness calculation), a tibial slope, a femoral sagittal flexion angle, a femoral external or internal rotation angle (e.g., optionally with the reference axis used for these angle measurements, such as posterior conylar axis, epicondylar axis), a design of patient specific guides, or the like.

In the example of a reverse or total shoulder arthroplasty, the intra-operative data elements may include an implant family, an implant brand, a selected implant component, a size, or length of a component (e.g., glenoid component, baseplate, augment, central, inferior, and superior screw, glenosphere, or the like), an instrumentation (e.g., reamers, impactors, etc.), a native or corrected glenoid version or inclination, native or corrected scapular axis (e.g., Friedman line), a glenosphere medio-lateral offset, a glenosphere inferior eccentricity, a position (e.g., anterior, posterior, medial, lateral) or orientation (e.g., inclination) of one or more of the various implant components, a percentage of contact between the implant and the bone, a deepest point of the glenoid, a superior screw SI angle, an inferior screw SI angle, a superior screw AP angle, an inferior screw AP angle, a design of patient specific guides, or the like.

In the example of a hip arthroplasty, the intra-operative data elements may include an implant family, an implant brand, a selected implant components, a size or length of a component, an acetabular or femoral anteversion or inclination, a leg length discrepancy, a femoral center of rotation, a change in center of rotation, a femoral offset (e.g., the distance between the acetabular center of rotation and the axis of the femoral implant), a combined or global offset, a varus or valgus angle of the femoral implant, a range of motion (which may be assessed by one or more of the following parameters: angles of flexion or extension, angles of adduction or abduction, the internal or external rotation of the leg), or the like.

For example, in a navigated or robotically guided knee or hip arthroplasty, the navigated or robotically guided surgical plan may be correlated with postoperative results including postoperative recovery protocols used to obtain the final results. With objective data obtained from a robotically guided procedure, a machine-learning model may be trained to assist in guiding selection of the best postoperative protocols to obtain positive outcomes. Patient mobile devices may be programmed to monitor and guide post-operative recovery protocols automatically.

Intraoperative collection of robotic surgical data may be used, in an example, with a final anatomy state (e.g., a final state of a knee, hip, or shoulder) along with postoperative outcomes to train a machine-learning postoperative protocol model for generating a postoperative plan. The intraoperative robotic data may be used as training data, labeled with positive or negative patient outcomes based on postoperative steps taken by patients.

Postoperative Data Collection

Postoperative recovery may be critical to positive long-term outcomes for orthopedic surgical procedures, but monitoring and controlling postoperative recovery has historically been a major challenge. Monitoring compliance with exercise regiments or limitations has relied primary on voluntary patient feedback, controlled therapy sessions, or periodic office visits. The lack of routine and reliable postoperative monitoring data makes adjusting postoperative recovery protocol recommendations difficult. Collection of postoperative patient data allows for development of a database of information that may be correlated to final outcomes, which allows for training of a machine-learning model to recommend protocols or protocol changes based on newly received post-operative patient data. The machine-learning model may provide risk stratification or risk classification of patients, for example with respect to risks of surgical complications, chances of post-operative success, or risks of failure to comply with prescribed pre- and post-operative therapy regimens.

Postoperative data collection may include the aforementioned data elements with respect to preoperative data collection, such as patient responses to questionnaires on their pain level (e.g., PROMs). In an example, a mymobility application may be used to collect this data, as described above. An Orthopedic Intelligence System may capture the surgeon and care team's impressions and conclusions as to whether the surgery was a success, or how certain procedural decisions could have been improved in hindsight. These healthcare provider outcome measurements may be used to modify or improve predictive or prescriptive analytics described herein.

In some examples, postoperative data collection includes data from implantable sensors, such as the Canary Health Implantable Reporting Processor (CHIRP) from Canary Medical, Inc. The CHIRP sensor is incorporated in the body of a permanent implant, such as the tibial stem of a knee replacement implant. Other locations for implantable sensors include the femoral stem of a hip implant system, acetabular cup of a hip implant system, or the glenoid, humerus, or glenosphere of an anatomic or reverse shoulder arthroplasty. The data from the sensor may complement or replace a step count, a range of motion, a qualitative gait analysis of a wearable sensor, or the like. The sensor data may provide further insight into potential complications from surgery, such as infection or loosening of the implant. This data on the outcome of the surgery, positive or negative, may be used as feedback in an analytics engine of the Orthopedic Intelligence System to train predictive or prescriptive analytics models. Implant sensor data may be used for fall detection in some examples.

System Outputs

For different tasks, different outputs may be generated by one or more machine learning models. For example, for a surgeon, an output may include a recommendation, likelihood of success, or other metric related to determining whether to select a patient, which patient to select, or a risk assessment for a patient pre-operatively. When a patient enrolls in the Orthopedic Intelligence System (such as by enrolling into the mymobility application) and optionally fills out some background information, the Orthopedic Intelligence System may give the surgeon a grading of the risk associated achieving the desired outcomes with the available treatment plans (e.g., in-patient/out-patient, total knee/partial knee, etc.).

For a patient or surgeon, it may be useful to know a predicted outcome of a surgical procedure. For example, a patient outcome prediction or recommendation may be provided from a machine learning trained model, such as seven days before the procedure. In this example, the Orthopedic Intelligence System may provide a prediction of the patient's outcome based on their history, preparation, procedure details, etc. When the surgeon determines that the predicted outcome is not desirable, the surgeon may determine whether the surgery should be delayed or changed, and what may be done to improve the predicted outcome (e.g., weight loss, stop smoking, etc.).

The predicted outcome may relate to expected cost, in some examples. In some of these examples, the predicted outcome may be used for preauthorization of a surgical procedure. A range or threshold cost prediction may be included in the predicted outcome (e.g., a likely upper or lower limit of costs, such as within a confidence interval). The cost information may be provided at particular points in a patient recovery timeline (e.g., at an initial consult, pre-operatively, shortly after a surgical procedure, after some recovery from the surgical procedure has occurred, or the like). In some examples, the cost information may be available continuously or on-demand (e.g., by a surgeon, a clinic, an insurance company, the patient, etc.). The cost prediction may be related to a likelihood of an audit or approval of a claim (e.g., by an insurance company or a government). A recommendation related to the cost prediction may include a change to a procedure, medical coding, or the like.

In some examples, the machine-learning model may provide an output related to surgical plan optimization. In these examples, the Orthopedic Intelligence System may provide patient-specific predictions for outcomes based on different target final knee (or hip, shoulder, spine, etc.) states. These target final knee states may be provided to the surgeon pre-operatively for use in developing a surgical plan, or to drive soft tissue optimization for example during a robotic surgery.

Other predictions or recommendations may be made using a machine learning trained model as described herein. For example, a slow post-op recovery detection or recommendation may be provided. In this example, patient recover may be tracked and when the patient is recovering more slowly than expected or desired, a recommendation for an intervention to improve the recovery trajectory may be output. The recommendation may include modifying a postoperative plan or timeline. For example, extending a timeline when the patient is not on track may alleviate the patient's anxiety, or improve the patient's motivation and allow the patient to not fall further behind.

In an example, a wearable may include a monitoring program that rewards a patient when the patient achieves a milestone or keeps on a timeline, completes exercises or education, or the like. The monitoring program may be used to provide rewards such as a discount or a monetary incentive. The discount or monetary incentive may be provided by an insurance company, in some examples, such as how gym memberships are often incentivized by insurance companies. Fraud may be prevented for these rewards by using gait information or heart rate information to perform checks on patient effort. In some examples, an implanted sensor may be used to verify rewards are to be disbursed.

In another example, a potential concern alert may be provided. In this example, an anomaly may be detected in a dataset for a patient (e.g., step count, heart rate, and PROMs score), signaling that the patient is at risk for an issue in the future.

In yet another example, a practice or surgical technique recommendation may be output. In this example, after a surgeon has a sufficient number of patients through a full episode of care in the system, data from those patients may be aggregated for a recommendation to the surgeon on surgical technique, care delivery metrics or feedback, or interventions to improve patient outcomes overall. In still another example, a postoperative protocol may be generated by a machine learning model for example based on an input of a final patient state or intraoperative patient data. The postoperative protocol may include rehabilitation, such as physical therapy or occupational therapy. The postoperative protocol may include education (e.g., recommended reading) for a patient, other types of therapy (e.g., heat or cold therapy), other recommended exercises or routines, or the like. Feedback may be provided during a postoperative protocol (which may optionally be fed back into a machine-learning algorithm to update a model), which may be used to update the postoperative protocol (e.g., via the model). Updates and feedback from a patient may be provided to a surgeon for training. In some examples, an aspect of improving patient outcomes includes preoperative patient data collection, assessment of preoperative patient state, or predictive analytics of patient outcomes.

In an example, a machine learning trained model may provide insight to a surgeon regarding billing practices. For example, for insurance reimbursement, some entries or types of entries may be more likely to be monitored or flagged. The model may use past claim audits to predict whether a particular entry or claim is likely to be audited. The model may output a recommendation to the surgeon, such as to suggest that the surgeon preemptively provide a reason or additional information related to the entry or claim, or to suggest that the surgeon obtain preauthorization for the procedure.

An example output may include a prediction or recommendation related to a cost of a procedure or timing of a procedure. The cost may be prospective for a patient, a surgeon, a clinic, an insurance company, a government, etc. The cost may be output as a prediction, or a recommendation may be generated to reduce a cost. In an example, the output may be based on a determination that a patient is unlikely to watch postoperative videos, for example because patient did not watch preoperative videos. In this example, the output may advise a physician that it is likely that there will be more in-person physical therapy, and thus additional costs for the patient may higher. The output may include a recommendation to the patient to watch the postoperative videos to decrease costs.

In some examples, an output may be based on a particular selected patient outcome. In these examples, a specialized model may be generated for selectable patient outcomes, or a generic model may include a patient outcome selectable as an input. Example selectable patient outcomes may include ability to perform a postoperative task or sequence of tasks, with an optional timeframe. For example, a patient may select “cycling,” as a desired outcome, for example within a month after a knee replacement procedure, or, the patient may select “golfing without pain” within six weeks after a rotator cuff procedure. In these examples, a model may be selected or configured to output an optimized plan (including a surgical procedure, where appropriate) for achieving the selected patient outcome. The particular patient outcomes may correspond to various objective measurements or goals, which may be identified from sensor data or model prediction. For the cycling example, knee joint and leg range of motion may be required to be within a particular range (e.g., full extension to 135 degrees flexion), and an optimized plan may be output to achieve the range, for example including physical therapy, a scheduled intervention, etc. The golf outcome may include a set of sub-outcomes, such as shoulder range of motion, walking short distances without pain, being upright for 5 hours, etc.

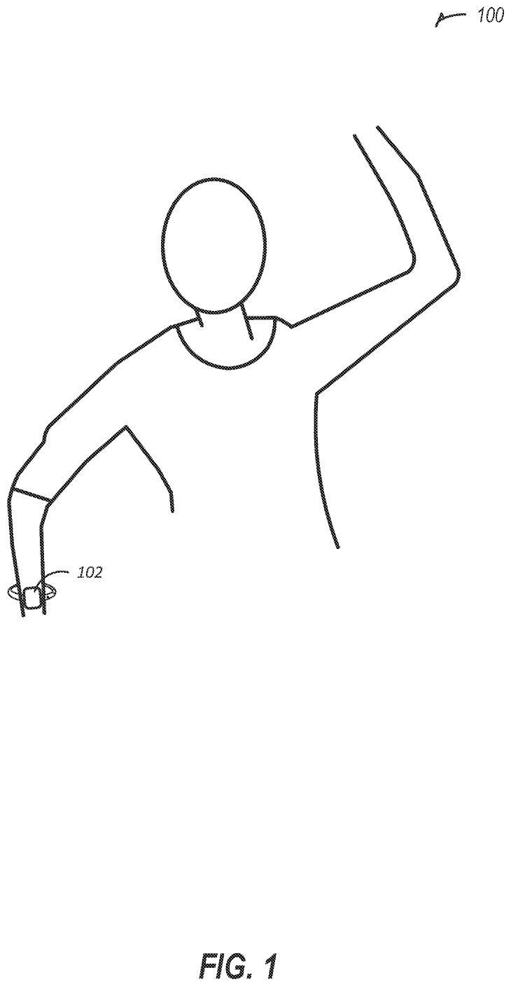

FIG. 1 illustrates a patient wearing a sensor device in accordance with at least one example of this disclosure. As discussed above, wearable technology, such as a watch, may be utilized to assist in obtaining objective measures related to patient activity as well as objective health related measurement (e.g., heart rate, body temperature, oxygen saturation levels, blood pressure, etc.). The following describes some specific example uses of a patient worn sensor device for collecting data for use within the Orthopedic Intelligence System.

The system 100 includes a first wrist-worn device 102 (e.g., a smart watch). The device 102 may be used as a standalone device, or may work in communication with a mobile phone. The device 102 may be used to gather data on steps, floors, gait, or the like. The device 102 may be used to deliver notification to a user. In certain examples, the user notifications are generated through the Orthopedic Intelligence System based on data received on patient performance pre-operatively or post-operatively.

In an example, the device 102 may be used to capture range of motion data or movement data, such as shoulder movement or range of motion data (e.g., adduction/abduction, flexion/extension, internal/external rotation), elbow movement or range of motion data (e.g., flexion/extension), wrist movement or range of motion data (e.g., pronation/supination), or the like. Range of motion data base be used pre-operatively to assist in recommending a surgical procedure or prediction outcomes. Post-operatively, range of motion data may assist in tracking recovery and providing recommendations on action a patient may take to obtain the desire outcome (e.g., exercises needed to reach a desire activity capability post-surgery).

Qualitative or quantitative data collection may be obtained. In an example for shoulder pain or a shoulder recommendation or procedure, raw range of motion (ROM) data may not be as valuable as describing the type of movement the patient is capable of or the pain in a patient. In an example, the device 102 may be used to extrapolate elbow information, for example, when a second sensor or device is not used near the elbow. In another example, the device 102 may be used near the elbow to detect elbow movement, pain, or range of motion data.

A picture or animation of the range of motion may be presented on a mobile device, such as the device 102 or a phone. For example, an animation may be sent as a small file format to illustrate the motion on the device 102. When a user clicks on a patient image, the user may be presented with the path of motion on the device 102.

FIG. 2 illustrates a wearable device in accordance with at least one example of this disclosure. The wearable device 202 may be in communication with a mobile device 204. The wearable device 202 may be a smart device, such as a smart watch, or other device with a user interface. Another example of a patient wearable device configured to capture objective measurement data for use in evaluating pre-operative and post-operative performance data related to a planned or completed surgical procedure.

In an example, the wearable device 202 includes a processor, memory, a display (e.g., for displaying a user interface), a transceiver (e.g., for communicating with the mobile device 204), and optionally an inertial measurement unit (IMU), a gyroscope, or an accelerometer.

The wearable device 202 may be used for advanced data collection. For example, the wearable device 202 may be used to measure stress response (e.g., heart rate) for example, as a proxy for pain during arm motions. These heartrate spikes may be tied to an animation to visualize the pain on the model. The wearable device 202 or other data collection device used with the systems and methods described herein may include one of a watch, fitness tracker, a wrist-worn device, a sweat monitor (e.g., electrolytes level), a blood-sugar monitor (e.g., for diabetes), a heart monitor (e.g., EKG or ECG), a heart rate monitor, a pulse oximeter, a stress level monitor (e.g., via a watch), a respiratory rate monitor device, a ‘life-alert,’ an ear wearable (e.g., for measuring intercranial pressure, such as via the tympanic membrane), a head attached wearable, an ultrasound wearable, a microphone dysphonia device, an augmented or virtual reality device (e.g., for measuring dizziness, gait, etc.), an implantable sensor, a traditional medical device implant, on-body, or attached devices (e.g., a dialysis machine, heart devices such as pacemaker, defibrillator, etc., CPAP, blood-sugar testing devices, drug tests, etc.), which may include internal, medically supervised, or at-home devices (three categories within the medical device category (ventilators, neurostim devices), a smart contact, a smart ring, a capillary refill test (e.g., for risk of sores or bruises, diabetic injuries), exercise equipment (e.g., elliptical, mirror, treadmill, bike like peloton, stair stepper, etc.), a phone app that tracks data, an intraoperative data collection device (e.g., vision and robotic information), a chest band (e.g., for respiration and heart rate), or the like.

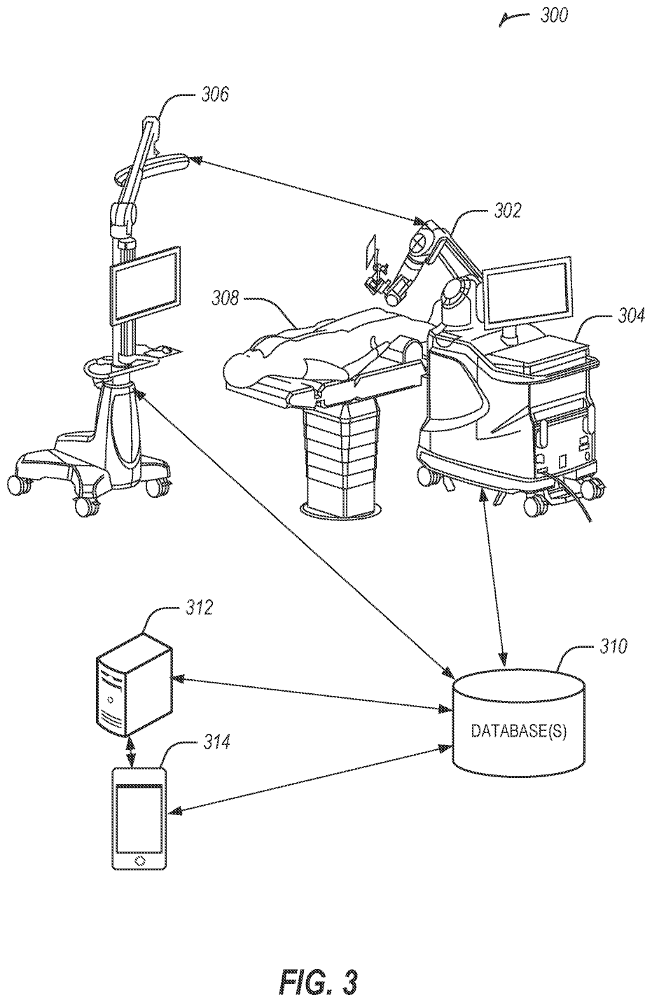

FIG. 3 illustrates a robotic surgery and feedback system 300 in accordance with some embodiments. The robotic surgery system may provide valuable objective data to the Orthopedic Intelligence System for predicting outcomes and training predictive models for future patients. Unlike a surgeon, a robotic surgical system, such as system 300, operates with measurable precision allowing for precise tracking of specific surgical parameters and correlation of objective intraoperative measurements with ultimate patient outcomes allowing for the Orthopedic Intelligence System to update predictive models to enhance future surgical procedures.

The system 300 includes a robotic surgical device 302, which may include a computing device 304 (e.g., a processor and memory for performing instructions). The system 300 includes a database 310. The robotic surgical device 302 may be used to perform a portion of a surgical procedure on a patient 308 (e.g., a partial or total knee arthroplasty, a hip arthroplasty, a shoulder arthroplasty, etc.). The robotic surgical device 302 (e.g., via the computing device 304) may store or send data, such as information about an action taken by the robotic surgical device 302 during the portion of the surgical procedure. The data may be sent to the database 310, which may be in communication with a server 312 or user device 314. In certain examples, the database 310 and server 312 are part of, or in communication with, the Orthopedic Intelligence System. The system may include a display device 306, which may be used to issue an alert, display information about a recommendation, or receive a request for additional information from a surgeon.

The system 300 may be used to generate or collect data pre-operatively or intra-operatively regarding aspects of a surgical procedure, such as actions taken by the robotic surgical device 302, input from a surgeon, patient anatomy information, or the like. The data may be saved, such as in the database 310, which may be accessed via a server 312 or a user device 314. Again, the data generated by the system 300 is uniquely objective and repeatable due to the nature of the robotic system, which means it may be effectively utilized by machine learning algorithms to improve surgical outcomes.

In an example, data generated or collected by the surgical robotic device 302 may include data relative to ordinary use of the surgical robotic device 302, data collected on robot use during a procedure, data on use of aspects of the system 300 such as, time spent by a user on a user interface, number of clicks or key presses on a user interface, an adjustment or change to a plan (e.g., pre-operative plan), differences between an original plan and a final plan, duration of use of the surgical robotic device 302, software stability or bugs, or the like.

Pre-operative data may include medical imaging of a target procedure site, statistical representations of bones involved in the procedure, virtual models generated of the target procedure site (e.g., a three-dimensional model of a knee joint (distal femur and proximal tibia)), planned implant position and orientation on the virtual models, and planned resections or similar operations to be performed, among other things. Intra-operative data may include soft tissue tension measurements of a joint, intra-operative adjustments to pre-operative plan (implant position/orientation and resections), actual resection parameters (e.g., position and orientation of resections on distal femur) and final implant location (in reference to known landmarks or pre-operative plan). Finally, post-operative data may include objective data obtained from follow up medical imaging or other mechanisms to assess implant performance or procedural success, but may also focus on subjective patient impressions and physical performance (e.g., range of motion and strength).

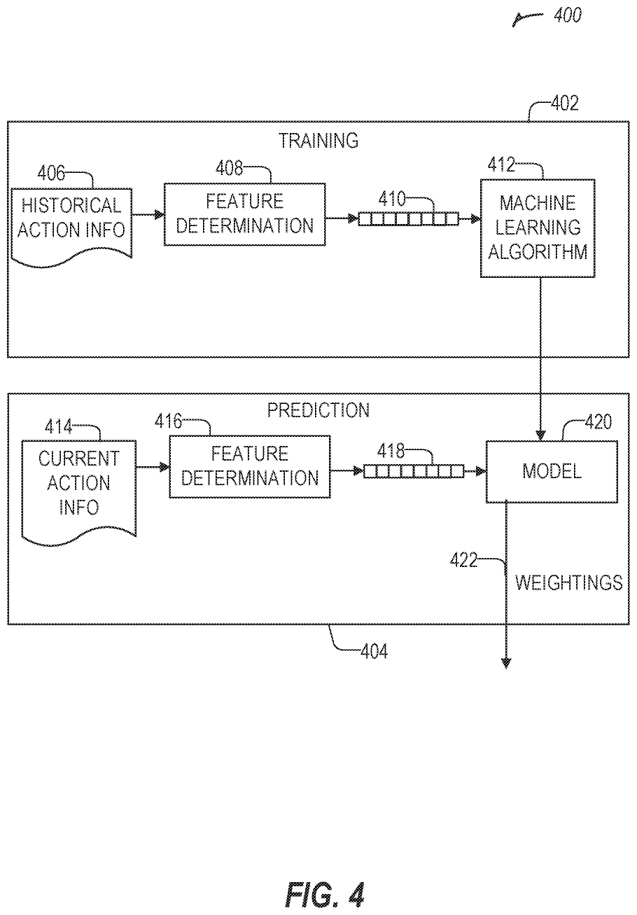

FIG. 4 illustrates a machine learning engine for determining feedback in accordance with some embodiments. The machine learning engine may be employed within the Orthopedic Intelligence System to recommend implants, procedures, surgical approaches, recovery routines, etc. A system may calculate one or more weightings for criteria based upon one or more machine learning algorithms. FIG. 4 shows an example machine learning engine 400 according to some examples of the present disclosure.

Machine learning engine 400 utilizes a training engine 402 and a prediction engine 404. Training engine 402 inputs historical information 406 for historical actions of a robotic surgical device, or stored or generated at a robotic surgical device, for example, into feature determination engine 408. Other historical information 406 may include preoperative data (e.g., comorbidities, varus/valgus data, pain, range of motion, or the like), intraoperative data (e.g., implant used, procedure performed, etc.), or postoperative data (e.g., range of motion, final state of patient anatomy, postoperative steps taken, such as physical therapy, education, etc., pain data, or the like). The historical action information 406 may be labeled with an indication, such as a degree of success of an outcome of a surgical procedure, which may include pain information, patient feedback, implant success, ambulatory information, or the like. In some examples, an outcome may be subjectively assigned to historical data, but in other examples, one or more labelling criteria may be utilized that may focus on objective outcome metrics (e.g., range of motion, pain rating, survey score, a patient satisfaction score, such as a forgotten knee score, a WOMAC score, shoulder assessment, hip assessment, or the like).

Feature determination engine 408 determines one or more features 410 from this historical information 406. Stated generally, features 410 are a set of the information input and is information determined to be predictive of a particular outcome. Example features are given above. In some examples, the features 410 may be all the historical activity data, but in other examples, the features 410 may be a subset of the historical activity data. The machine learning algorithm 412 produces a model 420 based upon the features 410 and the labels.

In the prediction engine 404, current action information 414 (e.g., preoperative data, a final state of patient anatomy, such as a final knee state, a surgical plan, an action to be taken or a last action taken, such as by a robotic surgical device, or the like) may be input to the feature determination engine 416. Feature determination engine 416 may determine the same set of features or a different set of features from the current information 414 as feature determination engine 408 determined from historical information 406. In some examples, feature determination engine 416 and 408 are the same engine. Feature determination engine 416 produces feature vector 418, which is input into the model 420 to generate one or more criteria weightings 422. The training engine 402 may operate in an offline manner to train the model 420. The prediction engine 404, however, may be designed to operate in an online manner. It should be noted that the model 420 may be periodically updated via additional training or user feedback (e.g., an update to a technique or procedure).

The machine learning algorithm 412 may be selected from among many different potential supervised or unsupervised machine learning algorithms. Examples of supervised learning algorithms include artificial neural networks, Bayesian networks, instance-based learning, support vector machines, decision trees (e.g., Iterative Dichotomiser 3, C4.5, Classification and Regression Tree (CART), Chi-squared Automatic Interaction Detector (CHAID), and the like), random forests, linear classifiers, quadratic classifiers, k-nearest neighbor, linear regression, logistic regression, and hidden Markov models. Examples of unsupervised learning algorithms include expectation-maximization algorithms, vector quantization, and information bottleneck method. Unsupervised models may not have a training engine 402. In an example embodiment, a regression model is used and the model 420 is a vector of coefficients corresponding to a learned importance for each of the features in the vector of features 410, 418.

Once trained, the model 420 may output a postoperative protocol for a patient based on a final state of patient anatomy, or pre- or intra-operative data. In another example, the model 420 may predict a postoperative protocol for a patient pre- or intra-operatively based on available data.

Data input sources for the model 420 may include one or more of a watch, fitness tracker, a wrist-worn device, a sweat monitor (e.g., electrolytes level), a blood-sugar monitor (e.g., for diabetes), a heart monitor (e.g., EKG or ECG), a heart rate monitor, a pulse oximeter, a stress level monitor (e.g., via a watch), a respiratory rate monitor device, a ‘life-alert,’ an ear wearable (e.g., for measuring intercranial pressure, such as via the tympanic membrane), a head attached wearable, an ultrasound wearable, a microphone dysphonia device, an augmented or virtual reality device (e.g., for measuring dizziness, gait, etc.), an implantable sensor, a traditional medical device implant, on-body, or attached devices (e.g., a dialysis machine, heart devices such as pacemaker, defibrillator, etc., CPAP, blood-sugar testing devices, drug tests, etc.), which may include internal, medically supervised, or at-home devices (three categories within the medical device category (ventilators, neurostim devices), a smart contact, a smart ring, a capillary refill test (e.g., for risk of sores or bruises, diabetic injuries), exercise equipment (e.g., elliptical, mirror, treadmill, bike like peloton, stair stepper, etc.), a phone app that tracks data, an intraoperative data collection device (e.g., vision and robotic information), a chest band (e.g., for respiration and heart rate), or the like.

Input data for the model 420 may include user input information, app data, such as from a food app, an exercise tracker, etc., a response to a questionnaire/PROM, video capture (e.g., for range of motion or strength), pain levels, opioid usage, compliance (e.g., with PT or OT or education steps), education data, exercise data, demographic or family history information, cognitive tests, BMI, exercise (daily/weekly/monthly), work status (unemployed, working, retired), age, gender, income/wealth status, children, marital status, or the like. The input data may include correlative data, such as including an innocuous or less offensive question as a proxy for a more difficult question (e.g., opioid usage). The proxy data may be more likely to be accurate (e.g., a patient may be more likely to lie about the proxied data). For example, innocuous or less invasive questions may include determining potential motivation of a patient from the patient's history, such as whether the patient has a previous cancer remission, employment at top of a field, previously ran a marathon, mountain climber, body builder, other difficult tasks faced in life that were overcome. Other input information to the model 420 may include clinician side data, a patient profile (e.g., demographics, preferences, etc.), a medical history of the patient, imaging, an arthritis lab panel, or the like.

An output for the model 420 may include a predictive functional outcome (e.g., a curve over time), such as with normalized ranges with thresholds/alerts (e.g., advanced gait metrics), a predictive PROMs outcome (e.g., score over time), a predictive return of ROM, a risk prediction or alert, such as for falls, readmission, infection, revision, DVT or other complication, a cost prediction (e.g., for office visits, readmission risk, inpatient/outpatient PT, infection), an appropriateness for telehealth follow up or in person follow up, a discharge location prediction (e.g., home, home with nurse visit, skilled nursing facility, home health, etc.), an appropriateness for virtual PT, a progression of virtual PT program, a recommendation for change in course of care, such as a shift to outpatient PT for virtual, wound consultation, manipulation, a recommended treatment plan (e.g., surgical or conservative), a supported co-decision making between patient and HCP, a recommendation for improved outcome based on modifiable risk factors, an output score and explanation of how/why the score was derived, or the like.

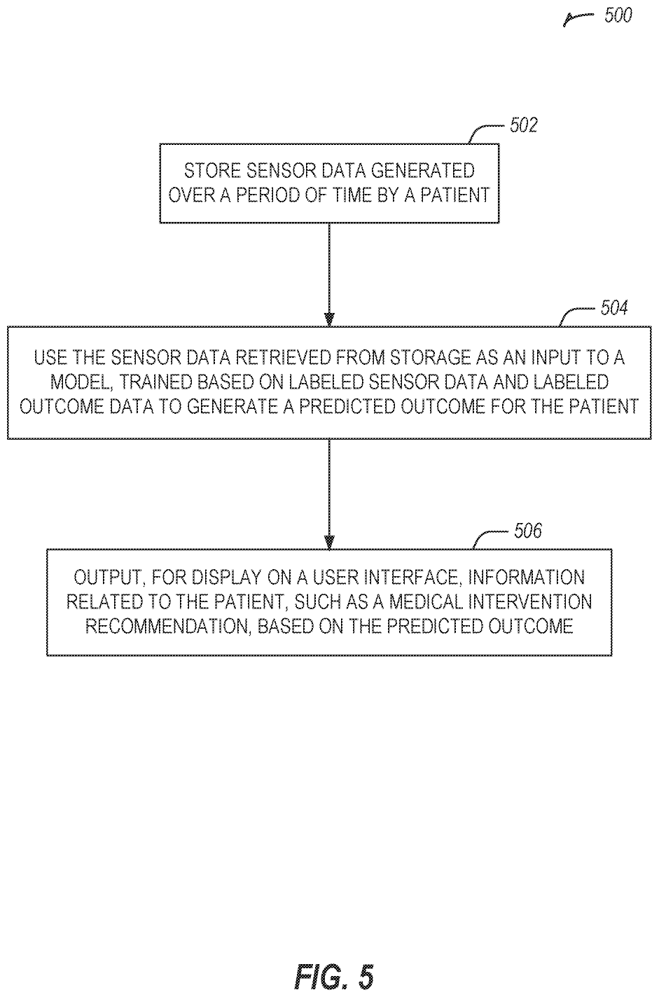

FIG. 5 illustrates a flowchart showing a technique 500 for providing a predicted outcome of a surgical intervention for a patient using the Orthopedic Intelligence System, in accordance with at least one example of this disclosure. The technique 500 is an example of functionality provide by the Orthopedic Intelligence System discussed herein.

The technique 500 includes an operation 502 to store sensor data generated over a period of time by a patient. The sensor data may originate from a wearable device, such as those discussed above.

The technique 500 includes an operation 504 to use the sensor data retrieved from storage as an input to a model, trained based on labeled sensor data and labeled outcome data to generate a predicted outcome for the patient.

The technique 500 includes an operation 506 to output, for display on a user interface, information related to the patient, such as a medical intervention recommendation, based on the predicted outcome.

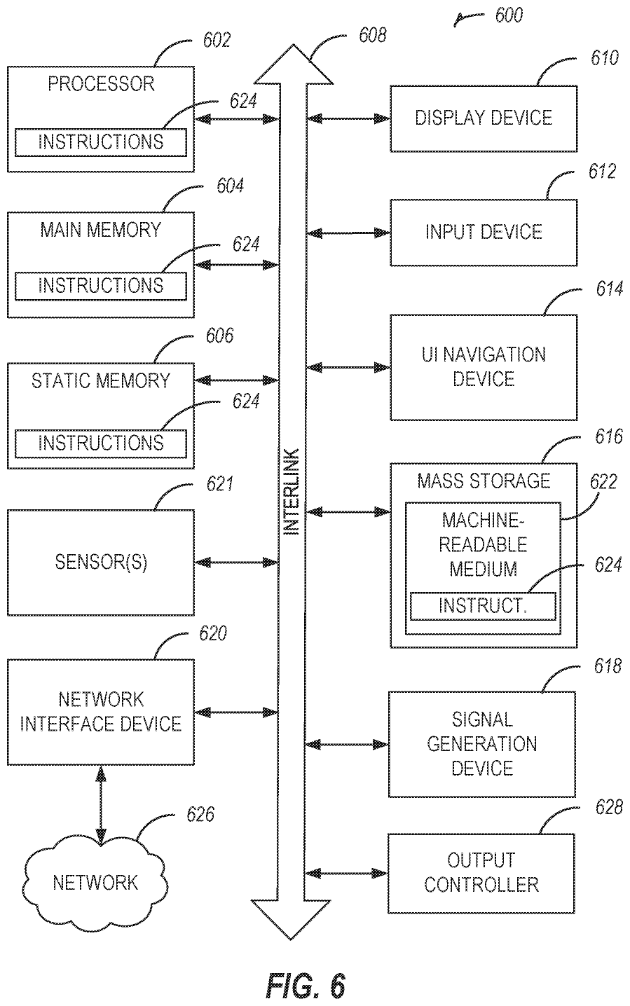

FIG. 6 illustrates a block diagram of an example machine 600 upon which any one or more of the techniques discussed herein may perform in accordance with some embodiments. This example machine may operate some or all of the Orthopedic Intelligence System discussed herein. In some examples, the Orthopedic Intelligence System may operate on the example machine 600. In other examples, the example machine 600 is merely one of many such machines utilized to operate the Orthopedic Intelligence System. In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610, an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., Universal Serial Bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium or multiple media (e.g., a centralized or distributed database, or associated caches and servers) configured to store the one or more instructions 624. The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

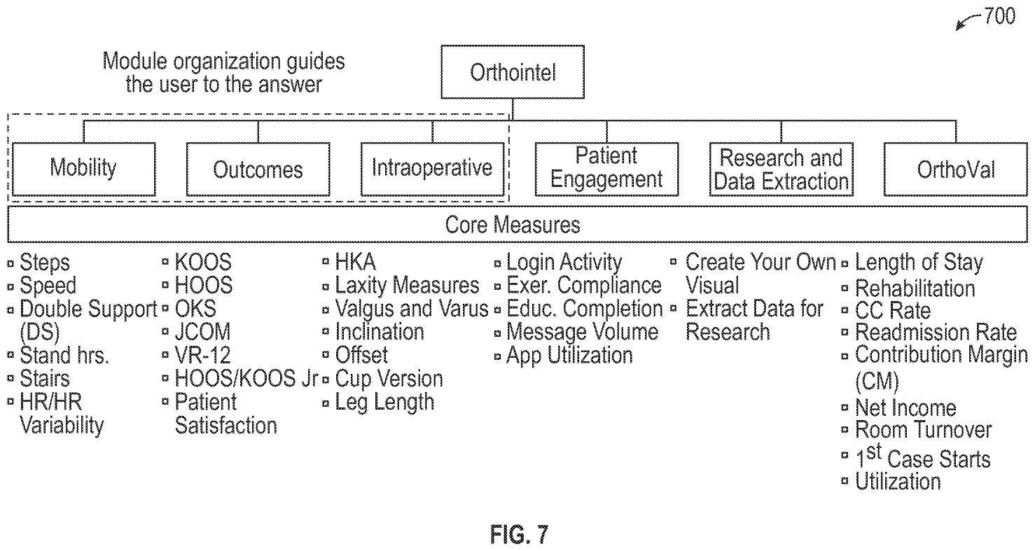

FIG. 7 illustrates an overview block diagram 700 for orthopedic interventions using the Orthopedic Intelligence System, in accordance with at least one example of this disclosure. The block diagram 700 illustrates functional blocks within the Orthopedic Intelligence System discussed throughout this disclosure. The overview block diagram 700 includes example investigative and analytic blocks, such as patient outcome, intraoperative feedback, patient feedback (e.g., mobility), patient engagement, research, or the like. The listed “core measures” include data inputs that may be used to generate the analytic information. Other example data input parameters are described herein. The overview block diagram 700 may use a machine learning model (e.g., trained according to the example shown in FIG. 4) to synthesize the input and output data.

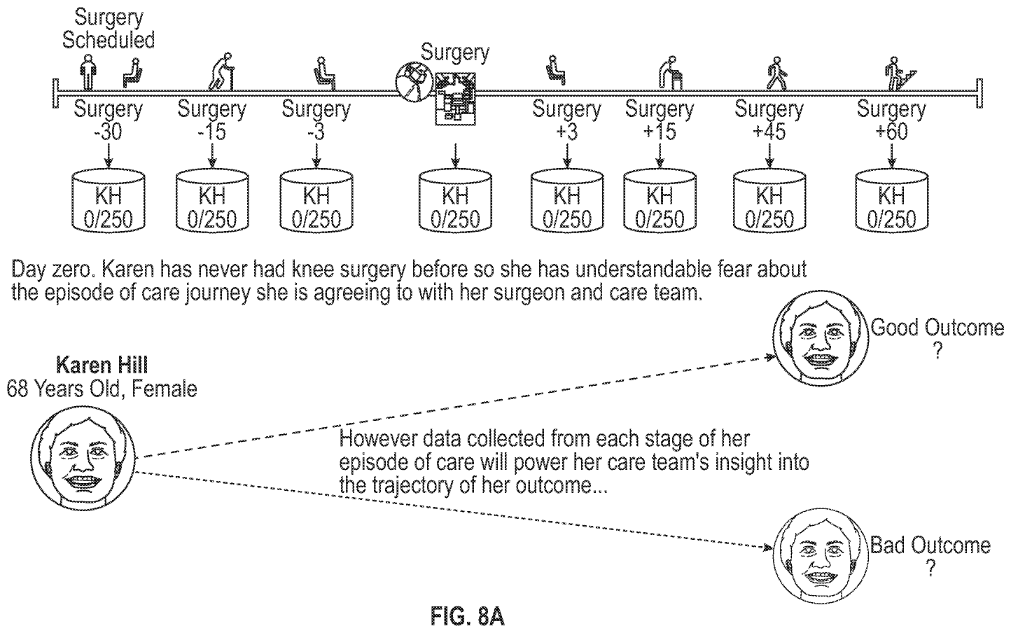

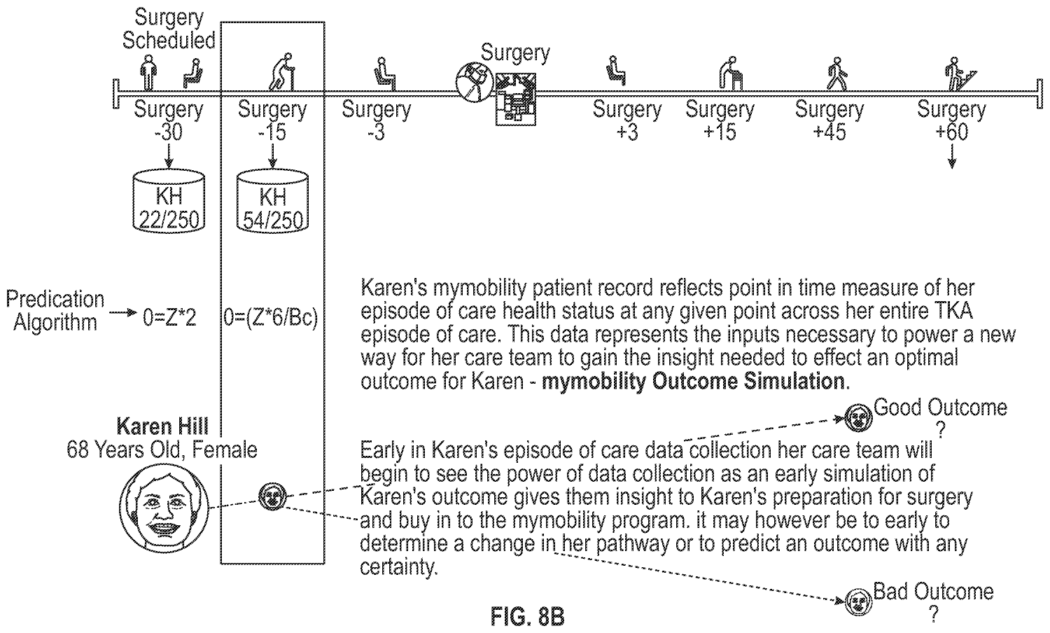

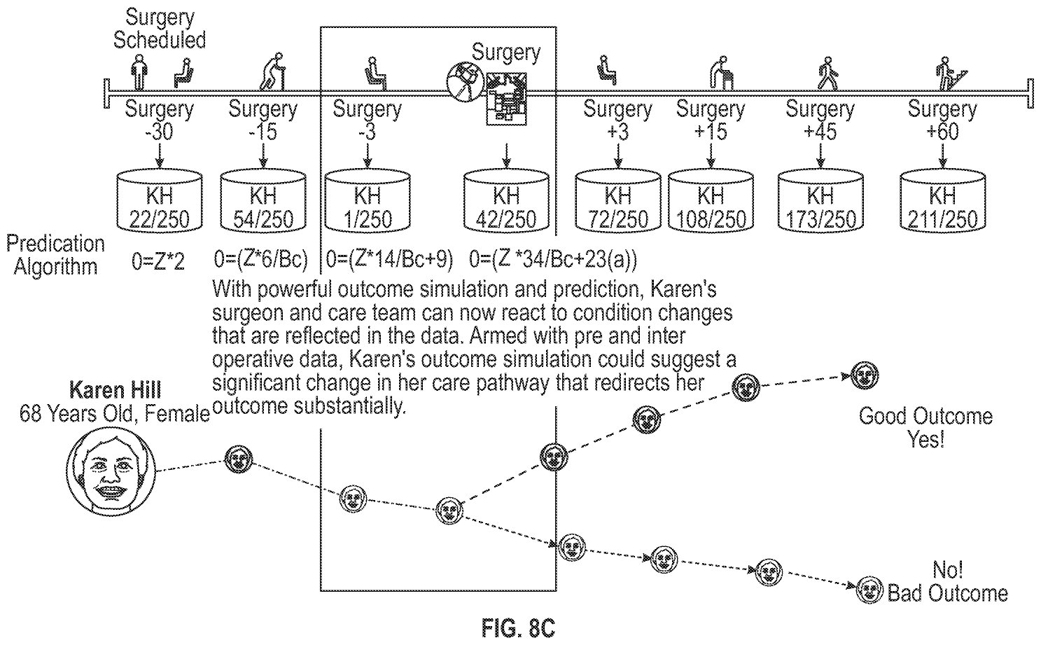

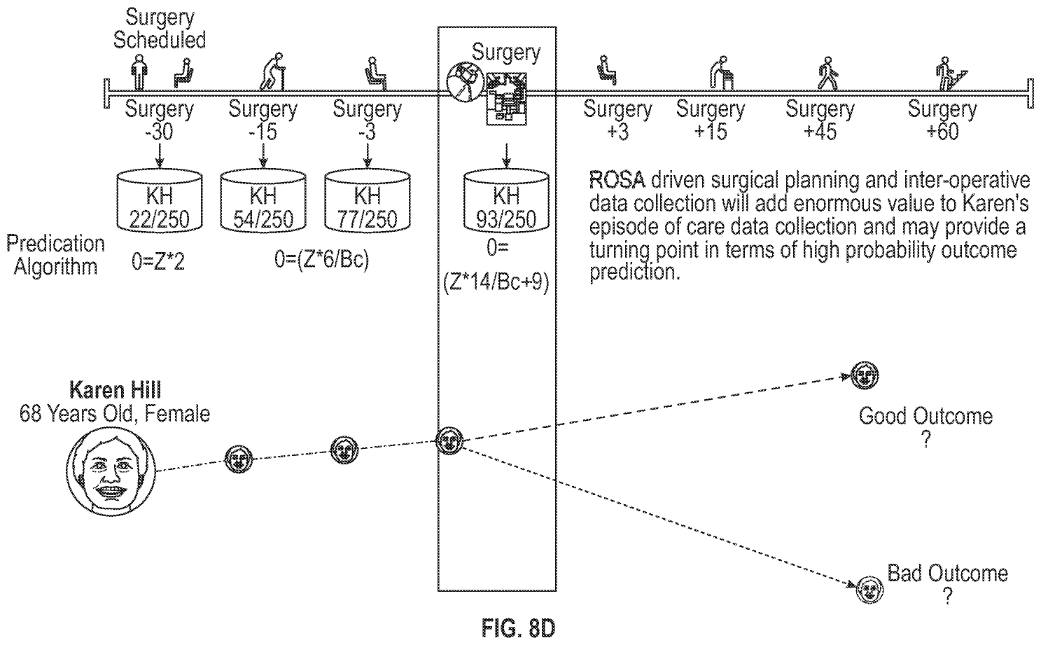

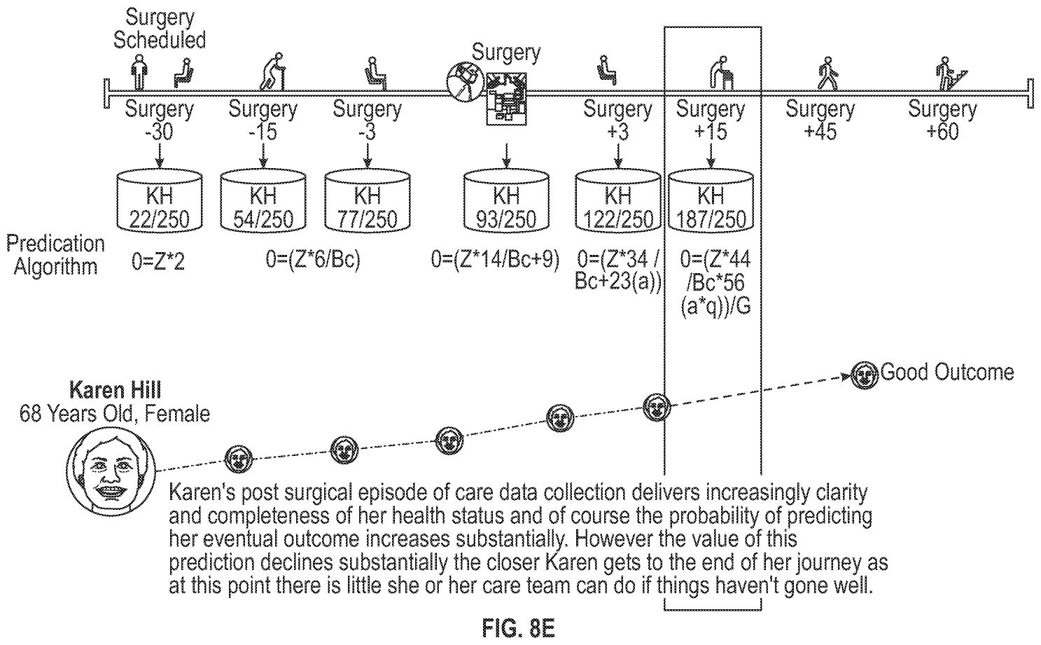

FIGS. 8A-8F illustrate example user interface timelines for a medical intervention guided by the Orthopedic Intelligence System, in accordance with at least one example of this disclosure. FIG. 8A shows an example patient, “Karen Hill” at an initial step of a treatment journey. At this point, the outcome is undetermined, but with additional data from Karen, as well as medical evaluation, a positive outcome vector may be achieved by consulting a trained model to generate a medical intervention prediction.

FIG. 8B shows that Karen is on a treatment journey related to an orthopedic procedure (e.g., a total knee arthroplasty or TKA) to relieve pain, discomfort, or lack of mobility. The treatment journey is shared between Karen and a care team (e.g., a nurse, a surgeon, an assistant, etc.), who may provide insights, answer questions, perform medical interventions (e.g., a surgical procedure), or the like.

FIG. 8C shows Karen's journey pre-operatively (e.g., from a few days or weeks before, up to the moment before surgery commences). Using a trained model to predict outcomes for Karen, the care team may predict which surgical interventions or choices made during surgery will lead to better outcomes for Karen. Utilizing the Orthopedic Intelligence System, inputs detailing Karen's physical condition may be processed to produce recommendations that may dramatically affect the outcome of her restorative surgical procedure. FIG. 8C illustrates how the prediction engine within the Orthopedic Intelligence System may intervene to change the trajectory of a patient's outcome.

FIG. 8D shows an example procedure that uses a surgical robot to aid in the surgical procedure. The surgical robot may provide unique pre-surgical planning opportunities or intra-operative data collection that may be used to generate a more positive outcome. The robotic pre-operative planning and intra-operative data collection may be used to improve post-operative outcomes for Karen.

FIG. 8E shows Karen's journey at an initial post-operative state. Use of data at this stage may be used to improve future surgical procedures for Karen or others, by adding labeling outcome data to pre- and intra-operative input data, such as for updating or improving a trained model.

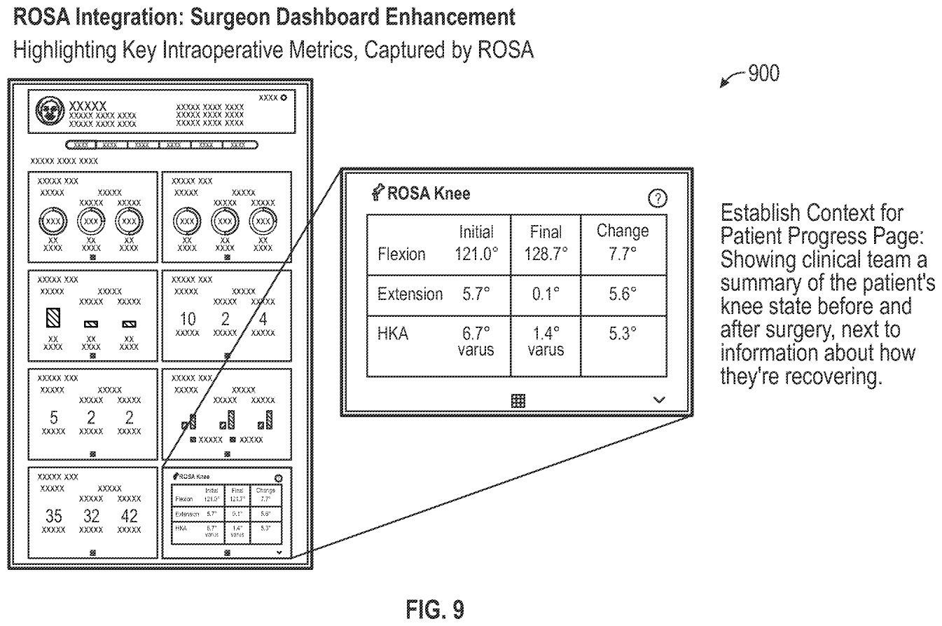

FIG. 9 illustrates a surgeon user interface 900 produced by the Orthopedic Intelligence System, in accordance with at least one example of this disclosure. The surgeon user interface 900 may be used to provide feedback to a surgeon (e.g., from a trained model), such as for predicting patient outcome or patient compatibility as described herein.

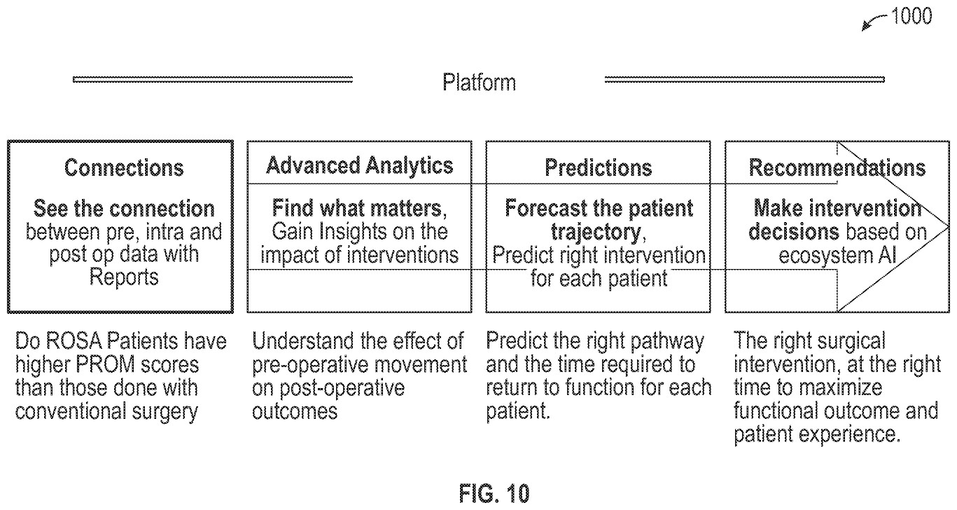

FIG. 10 illustrates an overview block diagram 1000 for feedback techniques utilized within the Orthopedic Intelligence System, in accordance with at least one example of this disclosure. The overview block diagram 1000 illustrates the analytics, predictions, and recommendations that may be presented to a surgeon (or other care team member) or a patient, based on the systems and techniques described herein.

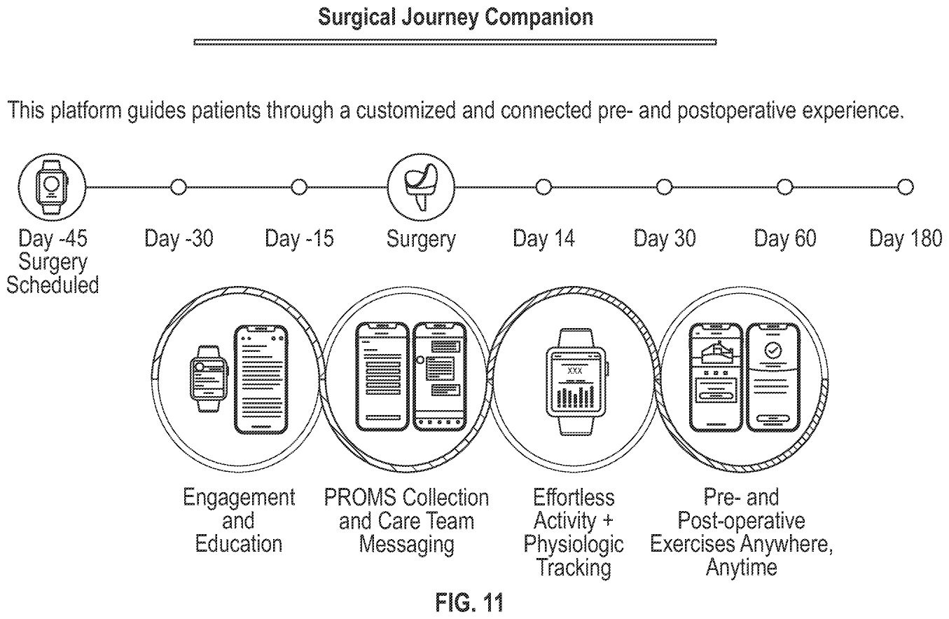

FIG. 11 illustrates an example surgical journey, for example from scheduling of a surgical intervention to the surgery itself, and beyond to post-operative recovery. An example of this journey for a patient may include accessing education information, performing exercises (e.g., physical therapy or occupational therapy), etc. In an example, a care team member may enroll the patient in a program configured to provide education and exercise information. The care team member or the patient may input demographics, select a care team or procedure, or assign a protocol. The patient may access engagement or education information, such as on a to-do list or via a notification before surgery. The program may include an encrypted communication component configured to allow the patient to securely communicate with the surgeon or other care team member. For example, the patient may discuss pain or concerns with a care team member. The program may provide video-guided exercises (e.g., post-surgery).

The surgical journey may be presented to a surgeon from a clinical perspective in an example, For the above example patient, the surgeon may be presented with demographic information, scheduling information, questions from the patient, etc. The surgeon may access patient progress (e.g., completed exercises, PT, OT, etc.), pain management, answer questions (e.g., using encrypted communication, such as via video, images, text, or audio, without needing an office visit by the patient), or export detailed patient reports for output to an electronic health record of the patient.

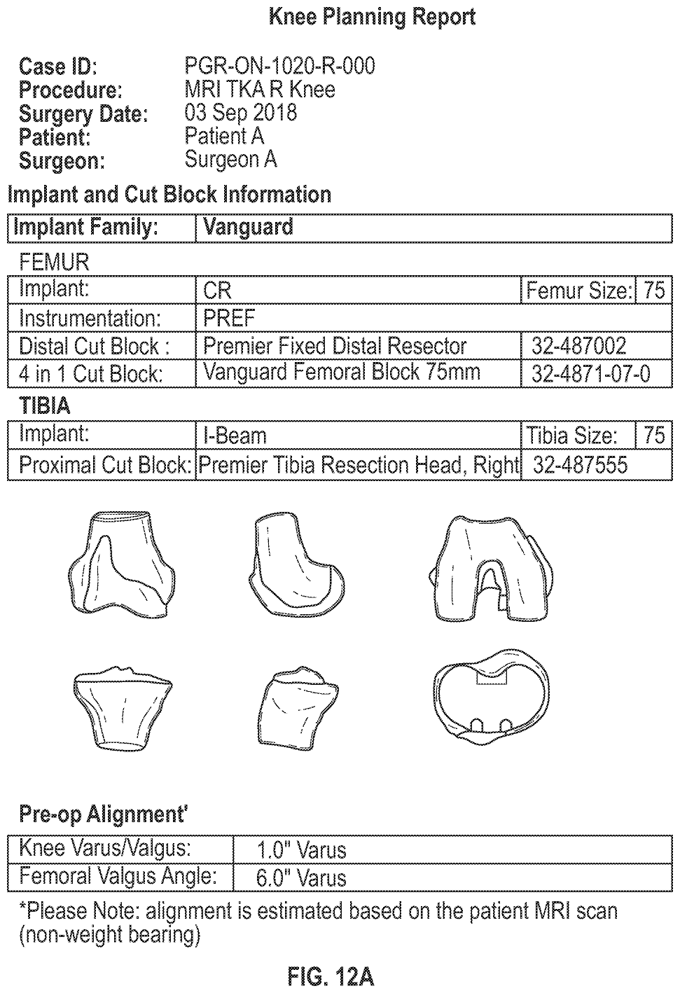

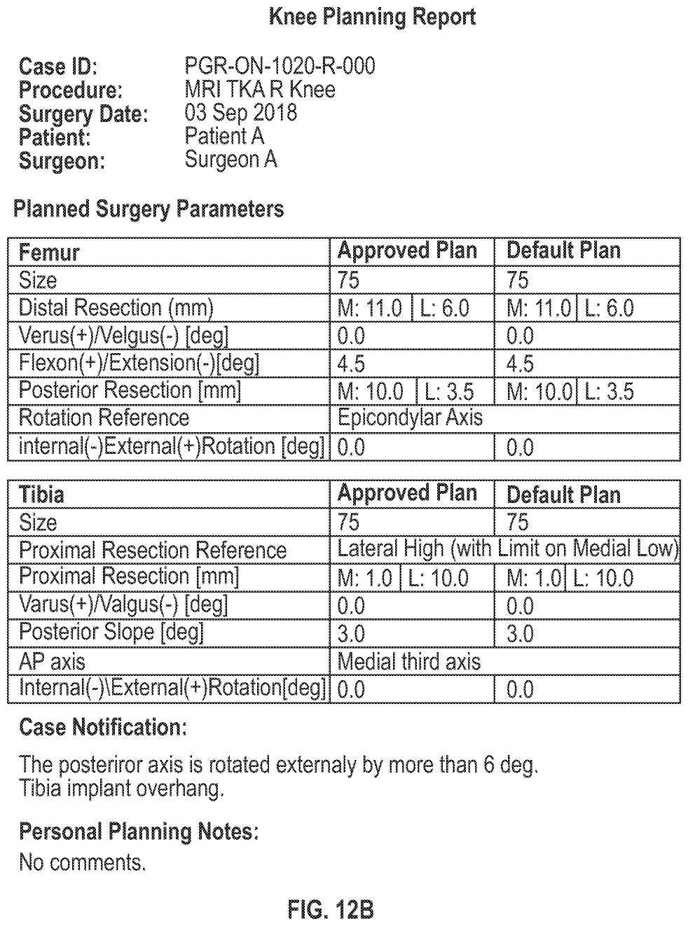

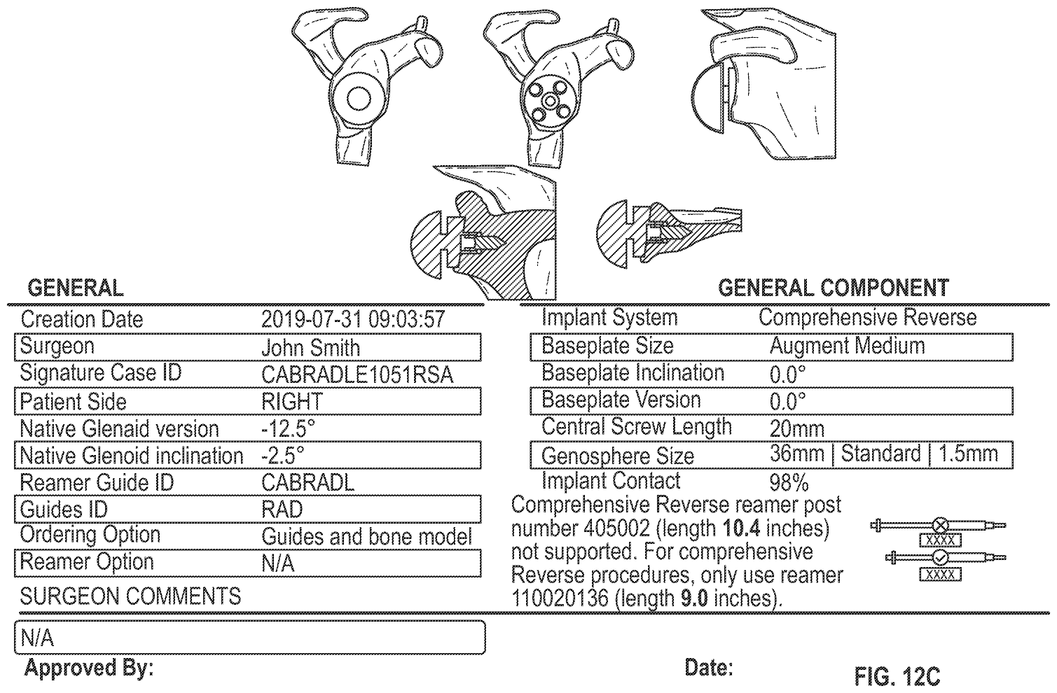

FIGS. 12A-12C illustrate example user interface information or reports for a surgeon generated by the Orthopedic Intelligence System, in accordance with at least one example of this disclosure. The example user interface information or reports of FIGS. 12A-12C include various patient-specific or surgery-specific details and data, such as implant size or location, cut recommendations, etc. A report may be generated based on predicted outcomes of a trained model, as described herein. The details or data of the report may be output by the model, or generated based on a predicted outcome. In an example, the model may output a set of potential outcomes (e.g., based on a selected intervention, such as a surgery, for example TKA or partial knee arthroplasty, or both knees at once versus one at a time, or other more granular decisions). A surgeon may select one or more of the potential outcomes to generate a report.

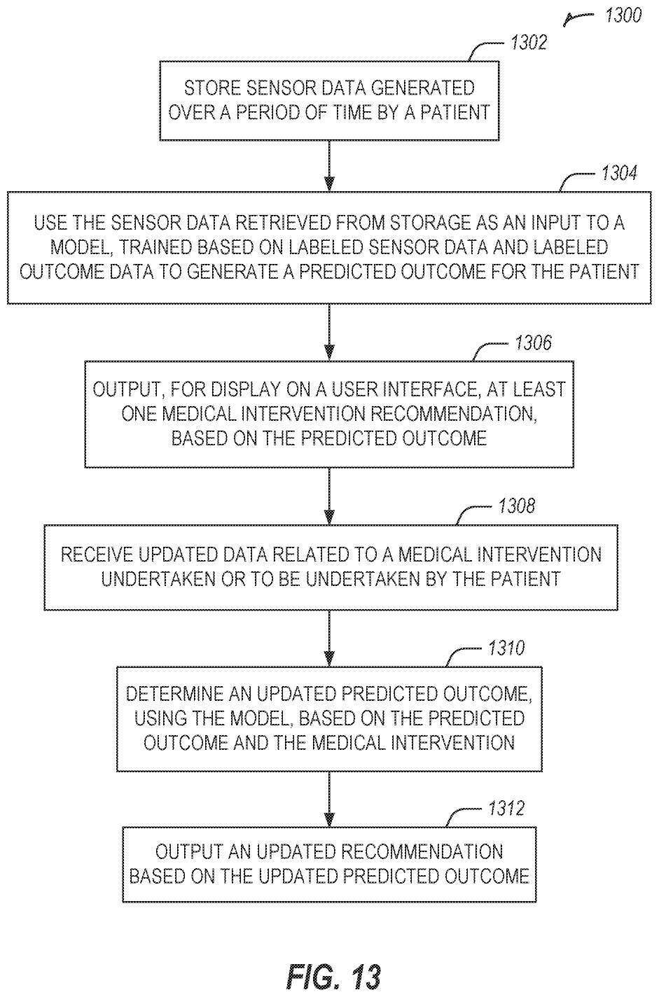

FIG. 13 illustrates a flowchart showing a technique 1300 performed by the Orthopedic Intelligence System for providing a predicted outcome of a surgical intervention for a patient in accordance with at least one example of this disclosure.

The technique 1300 includes an operation 1302 to store sensor data generated over a period of time by a patient. In an example, the sensor data may be generated by at least one of a wrist-worn device, a sweat monitor, a blood-sugar monitor, a heart monitor, a pulse oximeter, an ear-worn device, a head-attached device, an ultrasound wearable device, an augmented or mixed reality device, an implanted sensor, a medical device, a smart contact, a smart ring, exercise equipment, a mobile phone (e.g., via an app or sensor of the mobile phone), watch or wrist-worn device, Sweat monitor —electrolytes level, blood-sugar monitor diabetes), heart monitor—EKG (ECG), heart rate monitor, stress level, respiratory rate monitor device (e.g., from pressure in artery), ‘life-alert’, ear-wearable (e.g., to measure intercranial pressure measurements, such as via the tympanic membrane), head attached wearable, ultrasound wearable, microphone dysphonia, augmented or mixed reality (AR/MR) or virtual reality (VR) device (for measuring dizziness, gait, etc.), traditional medical device implant, on-body, or attached devices (dialysis machine, heart devices such as pacemaker, defibrillator, etc., CPAP, blood-sugar testing devices, drug tests, etc.), internal, medically supervised, or at-home devices (e.g., ventilators, neurostim devices, etc.), smart contacts, smart ring, capillary refill test (e.g., for risk of sores or bruises, diabetic injuries), exercise equipment (elliptical, mirror, treadmill, bike like peloton, stair stepper, etc.), intraoperative data (e.g., vision or robotic information), chest band for respiration and heart rate, or the like.

The technique 1300 includes an operation 1304 to use the sensor data retrieved from storage as an input to a model, trained based on labeled sensor data and labeled outcome data to generate a predicted outcome for the patient. In an example, operation 1304 may occur in response to receiving a query to initiate a patient evaluation for the patient. The model may be trained using a surgeon-specific parameter for a surgeon of the patient, a clinical parameter, or a resource availability parameter. The surgeon-specific parameter may include a type of surgery that the surgeon does, a level of experience of the surgeon, a preferred patient parameter of the surgeon (e.g., a side of the body), etc. The clinical parameter may include a patient-based parameter for patient state, availability of timing, surgeon availability, risk level, or the like. The resource availability parameter may include robotic data, personal surgical instrument data, etc.

In an example, the model may be selected based on a surgeon preference from among a plurality of models, the plurality of models including at least one of a model for a high risk patient, a model for a low risk patient, a traditional model, a model based on historical data corresponding to the surgeon, a least invasive recommendation model, or a patient age based model, and wherein at least two of the plurality of models generate different predicted outcomes for the patient.

The predicted outcome may include at least one of a predictive functional outcome (curve over time) & normalized ranges with thresholds/alerts (advanced gait metrics), predictive PROMs outcome (score over time), predictive return of ROM, risk prediction & alerts, e.g., falls, readmission, infection, revision, DVT or other complication, cost prediction (office visits, readmission risk, inpatient/outpatient PT, infection), appropriateness for telehealth follow up vs in person, output score and explanation of how/why the score was derived, an output score as a single quotient, the single quotient being an integer and representing a plurality of outcome predictors, or the like. The cost prediction may include costs associated with delay of a procedure or costs associated with accelerating a procedure (e.g., performing the procedure later or sooner than a current plan or recommendation). The cost prediction may include a prediction related to costs to a surgeon, costs to an insurance company, costs to a patient, costs to a clinic, or the like. The predicted outcome may be generated for a cost prediction based on legal, structural, or timing constraints, such as Medicare paying a bundled price or an insurance company offering incentives for an accelerated treatment based on a predicted improved outcome or fewer days in recovery, for example. The predicted outcome for cost prediction may include a predicted gain or loss for the patient, the surgeon, a clinic, an insurance company, the government (e.g., Medicare), etc.

The predicted outcome may include an outcome for conventional intervention (e.g., a low risk intervention) and a recommended intervention (e.g., a higher risk intervention). The predicted outcome may include manipulation under anesthesia, wherein the medical intervention undertaken includes breaking up a scar tissue, and wherein the updated recommendation includes a less invasive or non-surgical intervention than the at least one medical intervention recommendation. The predictive outcome may include early warnings that the patient's current post-operative trajectory indicates a risk for a manipulation under anesthesia. An updated recommendation may include at least one less invasive measure to avoid the need for a manual manipulation under anesthesia.

The technique 1300 includes an operation 1306 to output, for display on a user interface, at least one medical intervention recommendation, based on the predicted outcome. The user interface may be displayed on a mobile device of the patient that is communicatively coupled to a sensor that generated at least a portion of the sensor data. In this example, the technique 1300 may include displaying, to the patient on the user interface, a visualization of the at least one medical intervention recommendation being performed, the visualization including at least one of an image, a video, a 3D model, or a 4D model. In another aspect of this example, the technique 1300 may include displaying, to the patient on the user interface, an intervention timeline, a recovery period and an interactive user interface element configured to allow the patient to modify an input parameter to obtain a new predicted outcome based on the modification. When the new predicted outcome is unchanged from the predicted outcome, the technique 1300 in this aspect may include providing, on the user interface, a recommended parameter change for the patient, wherein the recommended parameter change results in a recommended predicted outcome that differs from the predicted outcome.

In an example, the at least one medical intervention recommendation includes at least one of a: discharge location prediction (e.g., home, skilled nursing facility, home health, etc.), recommendation for change in course of care—shift to outpatient PT for virtual, wound consultation, manipulation, recommended treatment plan (surgical or conservative), supported co-decision making between patient and HCP, recommendations for improved outcome based on modifiable risk factors, appropriateness for virtual PT, progression of virtual PT program, or the like.

The technique 1300 includes an operation 1308 to receive updated data related to a medical intervention undertaken or to be undertaken by the patient. For example, in a predictive mode, operation 1308 may receive updated data related to a selected medical intervention to be undertaken. In another example, operation 1308 may receive data related to a medical intervention that has already occurred. The technique 1300 includes an operation 1310 to determine an updated predicted outcome, using the model, based on the predicted outcome and the medical intervention. The technique 1300 includes an operation 1312 to output an updated recommendation based on the updated predicted outcome. In an example, the sensor data is generated by at least two wearable devices of the patient. In this example, operation 1312 may include outputting an indication of a data stream of the sensor data most responsible for the predicted outcome.

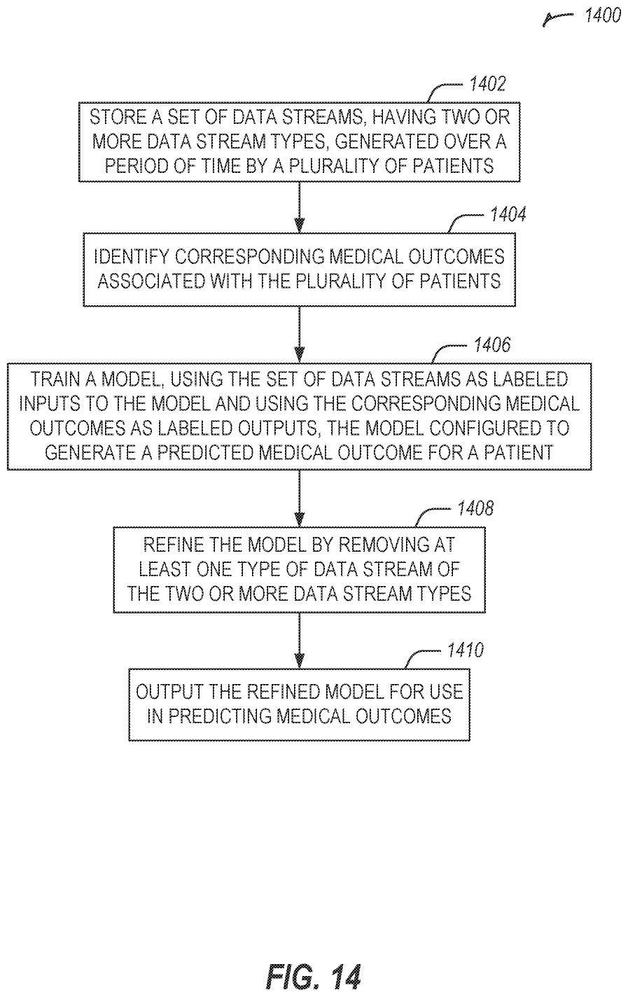

FIG. 14 illustrates a flowchart showing a technique 1400 performed within the Orthopedic Intelligence System for refining a trained model in accordance with at least one example of this disclosure. As discussed above, a benefit of utilizing a machine learning based system, such as the Orthopedic Intelligence System, is the ability for the system to continually update prediction models through objective and subjective data gather pre-operatively, intra-operatively and post-operatively. Outcome data is particularly important, especially when it may be connected back to objective measures and care decisions made pre-operatively and intra-operatively.

The technique 1400 includes an operation 1402 to store a set of data streams, having two or more data stream types, generated over a period of time by a plurality of patients. The technique 1400 includes an operation 1404 to identify corresponding medical outcomes associated with the plurality of patients. The technique 1400 includes an operation 1406 to train a model, using the set of data streams as labeled inputs to the model and using the corresponding medical outcomes as labeled outputs, the model configured to generate a predicted medical outcome for a patient.

The technique 1400 includes an operation 1408 to refine the model by removing at least one type of data stream of the two or more data stream types. Refining the model may include using a principal component analysis (PCA). The refinement may be determined without losing integrity of the results in the model. In an example, operation 1408 may include determining that a threshold minimum number of predicted outcomes remain unchanged when removing a data stream. The model may be further refined by a third party for example for use in a real-time prediction.

The technique 1400 includes an operation 1410 to output the refined model for use in predicting medical outcomes. Operation 1410 may include encrypting the refined model and outputting the encrypted refined model, for examples with a digital rights management attribute to prevent alterations to the refined model.

The technique 1400 may include an operation to validate the refined model using actual patient data and corresponding outcomes, the refined model matching predicted outcomes to the corresponding outcomes within a tolerance range. The technique 1400 may include determining outcome prediction differences between the model and the refined model based on removing the at least one type of data stream, and outputting information related to the outcome prediction differences. In this example, the technique 1400 may include scrapping the refined model when the outcome prediction differences transgress a threshold. The technique 1400 may further include receiving a query from a user of the encrypted refined model including a hash, validating the hash, and sending a response to the query indicating that the encrypted refined model is validated.

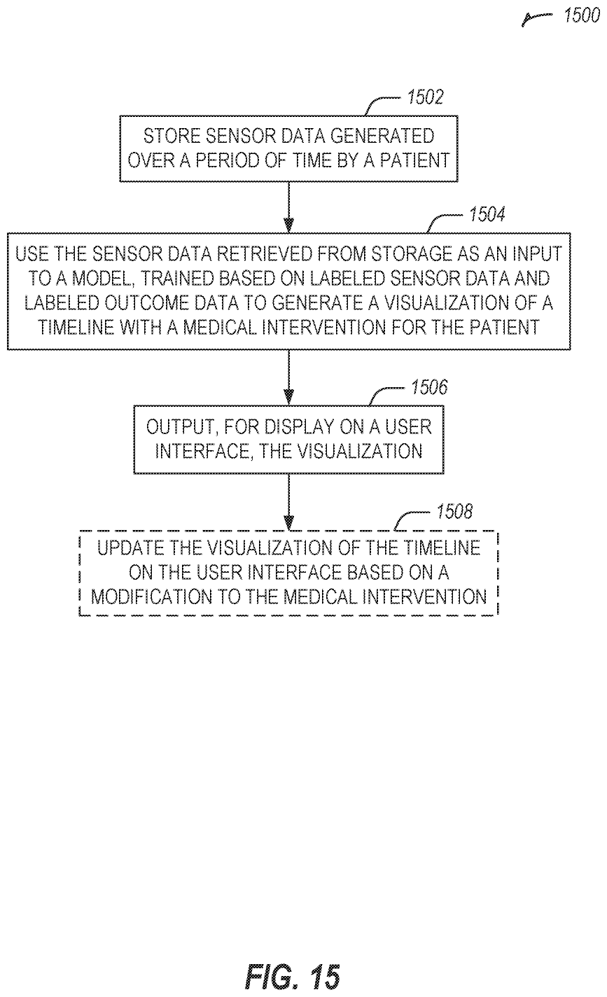

FIG. 15 illustrates a flowchart showing a technique 1500 performed by the Orthopedic Intelligence System for providing a visualization of a medical intervention timeline in accordance with at least one example of this disclosure. The technique 1500 includes an operation 1502 to store sensor data generated over a period of time by a patient.

The technique 1500 includes an operation 1504 to use the sensor data retrieved from storage as an input to a model, trained based on labeled sensor data and labeled outcome data to generate a visualization of a timeline with a medical intervention for the patient. In an example, operation 1504 may occur in response to receiving a query to initiate a patient evaluation for the patient. The timeline may include a recovery period after the medical intervention.

The technique 1500 includes an operation 1506 to output, for display on a user interface, the visualization. The user interface may be displayed on a mobile device of the patient that is communicatively coupled to a sensor that generated at least a portion of the sensor data.

The technique 1500 includes an optional operation 1508 to update the visualization of the timeline on the user interface based on a modification to the medical intervention. The visualization may include two timelines, a second of the two timelines including a robotic surgical intervention, wherein a recovery portion of the second timeline is shorter than a recovery portion of the timeline. The visualization may be updated based on generating, using patient data, including preoperative information, surgical information, and postoperative outcome data, as an input to a model, trained based on labeled historical patient data and labeled patient outcome data an updated visualization of the timeline of the patient based on the modification to the surgical intervention. The modification may be based on a complaint received by the patient, and wherein the updated visualization of the timeline includes an indication of a difference between a patient outcome score before and after the modification. In an example, the modification to the surgical intervention is identified to determine whether to change a traditional approach to patient treatment for patients having similar patient states to a patient state of the patient preoperatively. In an example, the modification is automatically determined using the model, the modification representing a different technique for performing the surgical intervention.

The technique 1500 may include receiving a modification to a patient state input and updating the visualization of the timeline on the user interface based on the modification to the patient state input. In an example, the technique 1500 includes providing, on the user interface, a recommended modification to a patient state input, and displaying a second visualization of the timeline based on the recommended modification.

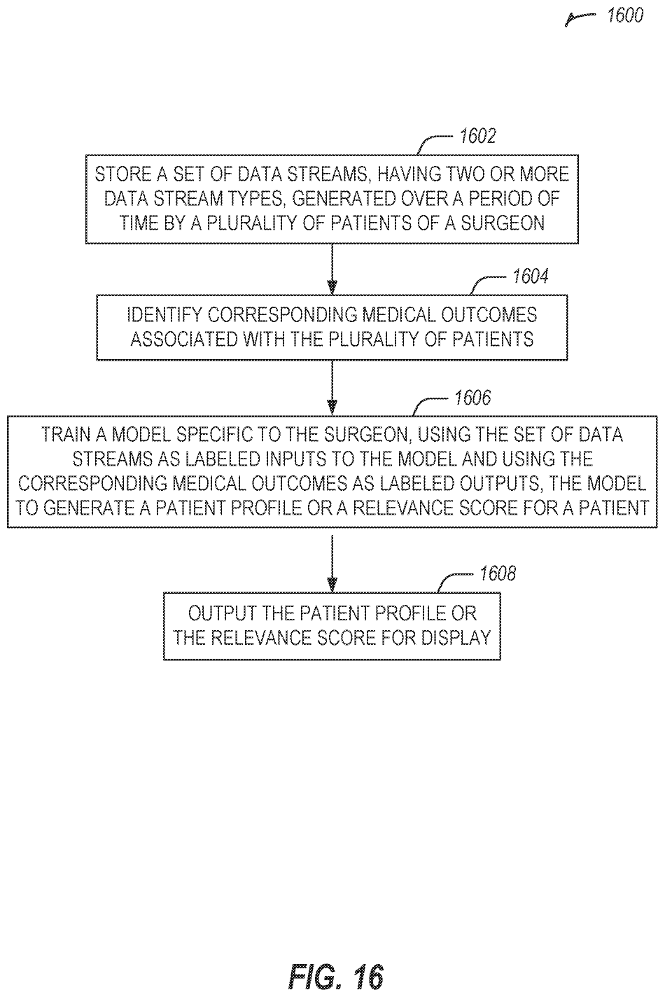

FIG. 16 illustrates a flowchart showing a technique 1600 performed by at least a portion of the Orthopedic Intelligence System for predicting a patient profile or relevance score in accordance with at least one example of this disclosure. The technique 1600 includes an operation 1602 to store a set of data streams, having two or more data stream types, generated over a period of time by a plurality of patients of a surgeon. The technique 1600 includes an operation 1604 to identify corresponding medical outcomes associated with the plurality of patients.

The technique 1600 includes an operation 1606 to train a model specific to the surgeon, using the set of data streams as labeled inputs to the model and using the corresponding medical outcomes as labeled outputs, the model to generate a patient profile or a relevance score for a patient. The patient profile may include a patient attribute for a patient, the patient attribute indicating a fit with a strategy or skill set of the surgeon.

The technique 1600 includes an operation 1608 to output the patient profile or the relevance score for display. The relevance score may be generated by inputting patient information for a patient to the model, in an example. The relevance score may indicate whether the patient is a fit with a strategy or skill set of the surgeon. In an example, the surgeon indicative of previous surgeries performed by the surgeon is used as a side input to the model while training the model to make a generic model specific to the surgeon.

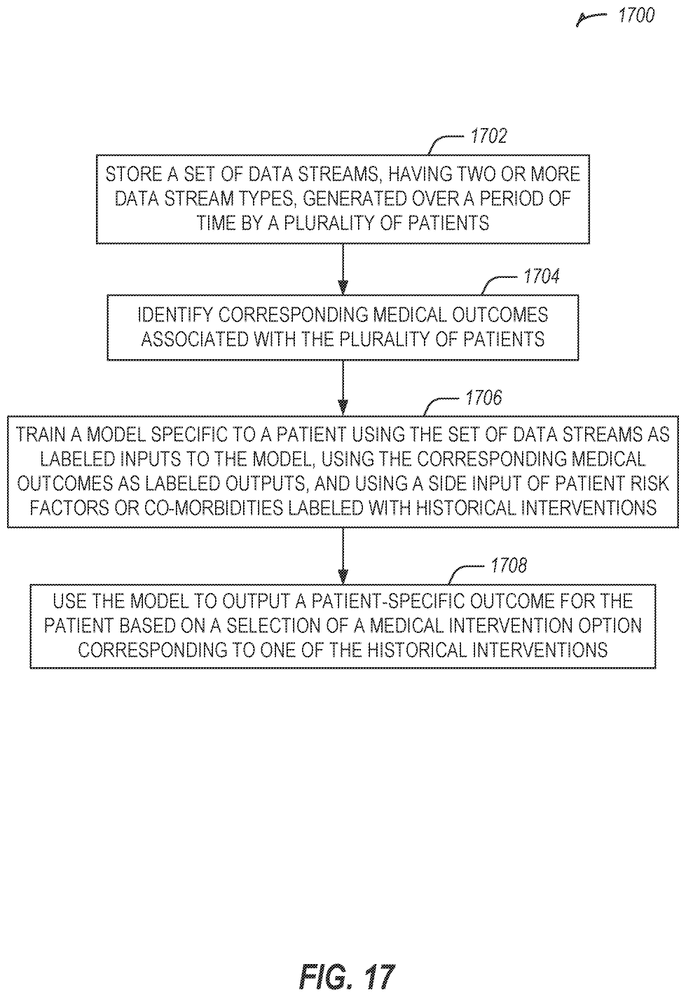

FIG. 17 illustrates a flowchart showing a technique 1700 performed by at least a portion of the Orthopedic Intelligence System for providing a predicted outcome of a surgical intervention for a patient in accordance with at least one example of this disclosure.

The technique 1700 includes an operation 1702 to store a set of data streams, having two or more data stream types, generated over a period of time by a plurality of patients. The technique 1700 includes an operation 1704 to identify corresponding medical outcomes associated with the plurality of patients. The technique 1700 includes an operation 1706 to train a model specific to a patient using the set of data streams as labeled inputs to the model, using the corresponding medical outcomes as labeled outputs, and using a side input of patient risk factors or co-morbidities labeled with historical interventions.

The technique 1700 includes an operation 1708 to use the model to output a patient-specific outcome for the patient based on a selection of a medical intervention option corresponding to one of the historical interventions. Operation 1708 may occur in response to receiving a selection of a medical intervention option corresponding to one of the historical medical interventions. Information related to the patient-specific outcome may be output for display.

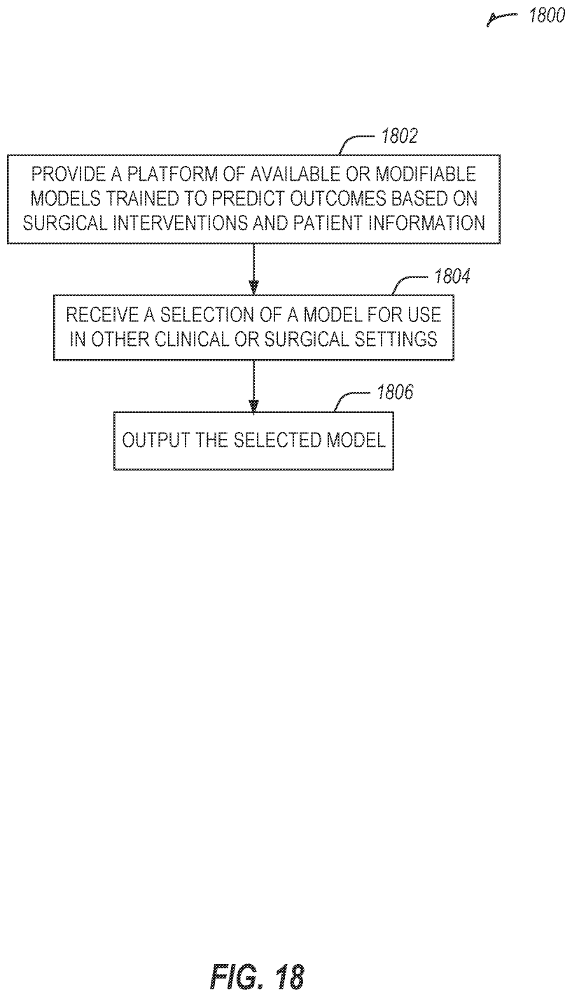

FIG. 18 illustrates a flowchart showing a technique 1800 performed by at least a portion of the Orthopedic Intelligence System for providing a platform including medical intervention models in accordance with at least one example of this disclosure. The technique 1800 includes an operation 1802 to provide a platform of available or modifiable models trained to predict outcomes based on surgical interventions and patient information.

The technique 1800 includes an operation 1804 to receive a selection of a model for use in other clinical or surgical settings. The technique 1800 includes an operation 1806 to output the selected model. In an example, the technique 1800 may include providing a trading platform for various models. Any particular surgeon may perform their own surgery, with their own models (which may be proprietary or may use the generic provided models, optionally modifiable). A surgeon may use the platform as a visualization and data storage/model usage, for example via an orthopedic software as a service (SaaS).

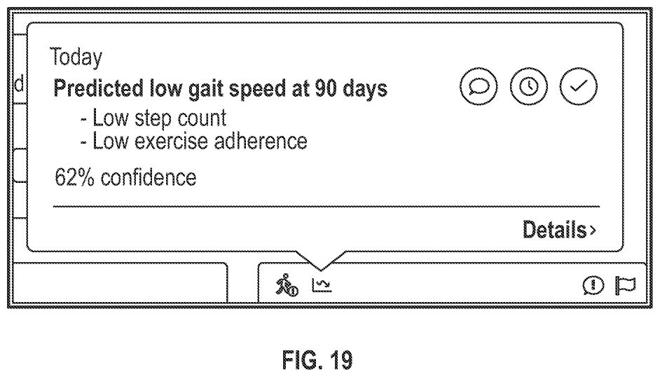

FIG. 19 illustrates an example user interface showing predictive support analytics information in accordance with at least one example of this disclosure. The example user interface of FIG. 19 may be used to provide a “grey-box” approach to machine learning or other model prediction. In this type of analytics, additional information is provided to support an output from a model, with the additional information including or based on how the output was generated by the model.

Typically, machine learning models use an “opaque-box” approach, where the input data is the learner data and the output fits defined definitions of predictions and recommendations, without any insights into how the output was derived from the input. The systems and techniques described herein include a partially transparent “grey-box” approach that bridges the medical research or literature-driven “transparent-box” approach (which lacks in speed, accuracy, etc.), with a pure machine learning “opaque-box” approach (which lacks in providing context or reasoning). The grey box approach allows the surgeon to use data features from the predictive modeling that are contextually meaningful. The grey box approach allows the surgeon to complement the machine learning outputs with their own experience and intuition, including optionally overriding the machine-generated conclusions. Feedback from the surgeon may be used to improve the model by providing feedback from the case with the “grey box” override. This approach allows for surgeon input into the feature extraction and feature selection of the Orthopedic Intelligence System's machine learning models, to complement other dimensionality reduction methods such as principal component analysis and isolate the variables that contribute to the tracked outcomes. The Orthopedic Intelligence System may survey surgeons as to their hypothesis as to which data elements are expected to be contextually meaningful, and these hypotheses may be tested by the machine learning algorithm and further refined.

In an example, the Orthopedic Intelligence System includes an input interface associated with the predictive output that allows a surgeon to provide output overrides that modifies the output of the prediction engine to better align the output with surgeon experiences or preferences. The Orthopedic Intelligence System may use the output overrides as further training input to update the models used by the predictive engine. Accordingly, the Orthopedic Intelligence System includes a built-in continual learning capability. In certain examples, the model updates may be surgeon-specific and not propagate throughout the entire Orthopedic Intelligence System. This feature allows individual surgeons to build a version of the system tailored to their practice preferences. Over time, the Orthopedic Intelligence System may monitor outcomes for the individual surgeon and compare those outcomes to other surgeons leading to opportunities for improvement of the entire system based on the success of an individualized system. In this example, the Orthopedic Intelligence System may send a request to a specific surgeon to allow system wide utilization of the prediction models used within the individualized system.

FIG. 19 includes an example grey box output, which provides a signal including “low step count” or “low exercise adherence” to supplement the base model output of “predicted low gait speed at 90 days,” and the likelihood of “62% confidence.” The low step count or low exercise adherence may be factors that went into the prediction of the low gait speed, or may be identified as potential reasons for the output. Said another way, the model may use the additional information to derive the prediction, or the model (or another model) may determine the additional information as a potential explanation for the prediction.

Each of the following non-limiting examples may stand on its own, or may be combined in various permutations or combinations with one or more of the other examples.

Example 1 is a method comprising: storing sensor data generated over a period of time by a patient, receiving a query to initiate a patient evaluation for the patient; in response to the query, using the sensor data retrieved from storage as an input to a model, trained based on labeled sensor data and labeled outcome data to generate a predicted outcome for the patient; outputting, for display on a user interface, at least one medical intervention recommendation based on the predicted outcome; receiving updated data related to a medical intervention undertaken by the patient; determining an updated predicted outcome, using the model, based on the predicted outcome and the medical intervention; and outputting an updated recommendation based on the updated predicted outcome.

In Example 2, the subject matter of Example 1 includes, wherein the sensor data is generated by at least two wearable devices of the patient, and further comprising outputting an indication of a data stream of the sensor data most responsible for the predicted outcome.

In Example 3, the subject matter of Examples 1-2 includes, wherein the model is trained using at least one of surgeon-specific parameters for a surgeon of the patient, clinical parameters, or resource availability parameters.

In Example 4, the subject matter of Examples 1-3 includes, wherein the user interface is displayed on a mobile device of the patient that is communicatively coupled to a sensor that generated at least a portion of the sensor data.

In Example 5, the subject matter of Example 4 includes, displaying, to the patient on the user interface, a visualization of the at least one medical intervention recommendation being performed, the visualization including at least one of an image, a video, a three-dimensional model, or a four-dimensional model.

In Example 6, the subject matter of Examples 4-5 includes, displaying, to the patient on the user interface, an intervention timeline, a recovery period and an interactive user interface element configured to allow the patient to modify an input parameter to obtain a new predicted outcome based on the modification.

In Example 7, the subject matter of Example 6 includes, wherein when the new predicted outcome is unchanged from the predicted outcome, further providing, on the user interface, a recommended parameter change for the patient, wherein the recommended parameter change results in a recommended predicted outcome that differs from the predicted outcome.

In Example 8, the subject matter of Examples 1-7 includes, wherein the sensor data is generated by at least one of: i. a wrist-worn device ii. a sweat monitor iii. a blood-sugar monitor iv. a heart monitor v. a pulse oximeter vi. an ear-worn device vii. a head-attached device viii. an ultrasound wearable device ix. an augmented or mixed reality device x. an implanted sensor xi. a medical device xii. a smart contact xiii. a smart ring xiv. exercise equipment or xv. a mobile phone.

In Example 9, the subject matter of Examples 1-8 includes, wherein the predicted outcome includes at least one of: a. Predictive Functional Outcome (curve over time) & normalized ranges with thresholds/alerts (advanced gait metrics) b. Predictive PROMs outcome (score over time) c. Predictive return of ROM d. Risk Prediction & alerts—Falls, Readmission, Infection, Revision, DVT or other complication e. Cost Prediction (office visits, readmission risk, inpatient/outpatient PT, infection) f. Appropriateness for telehealth follow up vs in person g. Output score and explanation of how/why the score was derived h. An output score as a single quotient, the single quotient being an integer and representing a plurality of outcome predictors.

In Example 10, the subject matter of Examples 1-9 includes, wherein the predicted outcome includes both outcomes for conventional intervention and a recommended intervention.

In Example 11, the subject matter of Examples 1-10 includes, wherein the at least one medical intervention recommendation includes at least one of a: a. Discharge location prediction—Home, Skilled Nursing facility, home health b. Recommendation for change in course of care—shift to outpatient PT for virtual, wound consultation, manipulation c. Recommended treatment plan (surgical or conservative) d. Supported co-decision making between patient and HCP e. Recommendations for improved outcome based on modifiable risk factors f. Appropriateness for virtual PT g. Progression of virtual PT program.

In Example 12, the subject matter of Examples 1-11 includes, wherein the predicted outcome includes manipulation under anesthesia, wherein the medical intervention undertaken includes breaking up a scar tissue, and wherein the updated recommendation includes a less invasive or non-surgical intervention than the at least one medical intervention recommendation.

In Example 13, the subject matter of Examples 1-12 includes, wherein the model is selected based on a surgeon preference from among a plurality of models, the plurality of models including at least one of a model for a high risk patient, a model for a low risk patient, a traditional model, a model based on historical data corresponding to the surgeon, a least invasive recommendation model, or a patient age based model, and wherein at least two of the plurality of models generate different predicted outcomes for the patient.

Example 14 is a method comprising: storing a set of data streams, having two or more data stream types, generated over a period of time by a plurality of patients; identifying corresponding medical outcomes associated with the plurality of patients; training a model, using the set of data streams as labeled inputs to the model and using the corresponding medical outcomes as labeled outputs, the model configured to generate a predicted medical outcome for a patient; using a principal component analysis (PCA) to refine the model by removing at least one type of data stream of the two or more data stream types; and outputting the refined model for use in predicting medical outcomes.

In Example 15, the subject matter of Example 14 includes, wherein the model is further refined by a third party for use in real-time prediction.

In Example 16, the subject matter of Examples 14-15 includes, validating the refined model using actual patient data and corresponding outcomes, the refined model matching predicted outcomes to the corresponding outcomes within a tolerance range.

In Example 17, the subject matter of Examples 14-16 includes, determining outcome prediction differences between the model and the refined model based on removing the at least one type of data stream, and outputting information related to the outcome prediction differences.

In Example 18, the subject matter of Example 17 includes, scrapping the refined model when the outcome prediction differences transgress a threshold.

In Example 19, the subject matter of Examples 14-18 includes, wherein outputting the refined model includes encrypting the refined model, and outputting the encrypted refined model with a digital rights management attribute to prevent alterations to the refined model.

In Example 20, the subject matter of Example 19 includes, receiving a query from a user of the encrypted refined model including a hash; validating the hash; sending a response to the query indicating that the encrypted refined model is validated.

Example 21 is an ear-worn device comprising: a sensor to capture data over a period of time; a communication component to: send a set of previously captured data from the period of time to a processing device; and receive instructions for outputting audio based on a medical intervention timeline determined by the processing device based on the set of previously captured data and a trained model; and a speaker to output audio based on the instructions.

Example 22 is a method comprising: storing sensor data generated over a period of time by a patient; receiving a query to initiate a patient evaluation for the patient; in response to the query, using the sensor data retrieved from storage as an input to a model, trained based on labeled sensor data and labeled outcome data to generate a visualization of a timeline with a medical intervention for the patient; outputting, for display on a user interface, the visualization; receiving a modification to the medical intervention; and updating the visualization of the timeline on the user interface based on the modification.

In Example 23, the subject matter of Example 22 includes, wherein the user interface is displayed on a mobile device of the patient that is communicatively coupled to a sensor that generated at least a portion of the sensor data.

In Example 24, the subject matter of Examples 22-23 includes, receiving a modification to a patient state input and updating the visualization of the timeline on the user interface based on the modification to the patient state input.

In Example 25, the subject matter of Examples 22-24 includes, wherein the timeline includes a recovery period after the medical intervention.

In Example 26, the subject matter of Examples 22-25 includes, providing, on the user interface, a recommended modification to a patient state input, and displaying a second visualization of the timeline based on the recommended modification.

In Example 27, the subject matter of Examples 22-26 includes, wherein the visualization includes two timelines, a second of the two timelines including a robotic surgical intervention, wherein a recovery portion of the second timeline is shorter than a recovery portion of the timeline.

Example 28 is a method comprising: outputting, for display on a user interface, a visualization of a historical timeline of a patient including a surgical intervention; receiving a modification on the user interface to the surgical intervention; generating, using patient data, including preoperative information, surgical information, and postoperative outcome data, as an input to a model, trained based on labeled historical patient data and labeled patient outcome data an updated visualization of the timeline of the patient based on the modification to the surgical intervention; and outputting the updated visualization of the timeline on the user interface.

In Example 29, the subject matter of Example 28 includes, wherein the modification to the surgical intervention is based on a complaint received by the patient, and wherein the updated visualization of the timeline includes an indication of a difference between a patient outcome score before and after the modification.

In Example 30, the subject matter of Examples 28-29 includes, wherein the modification to the surgical intervention is identified to determine whether to change a traditional approach to patient treatment for patients having similar patient states to a patient state of the patient preoperatively.

In Example 31, the subject matter of Examples 28-30 includes, wherein the modification to the surgical procedure is automatically determined using the model, the modification representing a different technique for performing the surgical intervention.

Example 32 is a method comprising: storing a set of data streams, having two or more data stream types, generated over a period of time by a plurality of patients of a surgeon; identifying corresponding medical outcomes associated with the plurality of patients; training a model specific to the surgeon, using the set of data streams as labeled inputs to the model and using the corresponding medical outcomes as labeled outputs, the model configured to generate, for the surgeon, a patient profile or a relevance score for a patient; outputting the patient profile or the relevance score for display.

In Example 33, the subject matter of Example 32 includes, wherein the patient profile includes a patient attribute for a patient, the patient attribute indicating a fit with a strategy or skill set of the surgeon.

In Example 34, the subject matter of Examples 32-33 includes, wherein the relevance score is generated by inputting patient information for a patient to the model, and wherein the relevance score indicates whether the patient is a fit with a strategy or skill set of the surgeon.

In Example 35, the subject matter of Examples 32-34 includes, wherein surgeon data indicative of previous surgeries performed by the surgeon is used as a side input to the model while training the model to make a generic model specific to the surgeon.

Example 36 is a method comprising: storing a set of data streams, having two or more data stream types, generated over a period of time by a plurality of patients; identifying corresponding medical outcomes associated with the plurality of patients; training a model specific to a patient, by: using the set of data streams as labeled inputs to the model and using the corresponding medical outcomes as labeled outputs; and using a side input of patient risk factors or co-morbidities labeled with historical medical interventions; receiving a selection of a medical intervention option corresponding to one of the historical medical interventions; using the model to output a patient-specific outcome for the patient based on the selection; and outputting information related to the patient-specific outcome for display.

Example 37 is a method comprising: providing a platform of available or modifiable models trained to predict outcomes based on surgical interventions and patient information; receiving a selection of a model for use in other clinical or surgical settings; and outputting the selected model.

Example 38 is at least one machine-readable medium including instructions that, when executed by processing circuitry, cause the processing circuitry to perform operations to implement of any of Examples 1-37.

Example 39 is an apparatus comprising means to implement of any of Examples 1-37.

Example 40 is a system to implement of any of Examples 1-37.

Example 41 is a method to implement of any of Examples 1-37.

Example 42 is a method, system, machine-readable medium, or apparatus comprising elements of one or more of Examples 1-37.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

What is claimed is:

1. A method comprising:
storing sensor data, the sensor data generated over a period of time by a patient;
receiving a query to initiate a patient evaluation for the patient;
in response to the query, using the sensor data retrieved from storage as an input to a machine learning model, the machine learning model trained based on labeled sensor data and labeled outcome data and the machine learning model configured to generate a predicted outcome for the patient;
outputting, for display on a user interface, at least one medical intervention recommendation based on the predicted outcome;
receiving updated data related to a medical intervention undertaken by the patient;
determining an updated predicted outcome, using the machine learning model, based on the predicted outcome and the medical intervention; and
outputting an updated recommendation based on the updated predicted outcome.

2. The method of claim 1, wherein the sensor data is generated by at least two wearable devices of the patient, and further comprising outputting an indication of a data stream of the sensor data most responsible for the predicted outcome.

3. The method of claim 1, wherein the machine learning model is trained using at least one of surgeon-specific parameters for a surgeon of the patient, clinical parameters, or resource availability parameters.

4. The method of claim 1, wherein the user interface is displayed on a mobile device of the patient that is communicatively coupled to a sensor that generated at least a portion of the sensor data.

5. The method of claim 4, further comprising displaying, to the patient on the user interface, a visualization of a surgical intervention corresponding to the at least one medical intervention recommendation, the visualization including at least one of an image, a video, a three-dimensional model, or a four-dimensional model.

6. The method of claim 4, further comprising displaying, to the patient on the user interface, an intervention timeline, a recovery period, and an interactive user interface element configured to allow the patient to modify an input parameter to obtain a new predicted outcome based on the modification via the machine learning model.

7. The method of claim 6, further comprising, when the patient modification to the input parameter does not change the predicted outcome, providing, on the user interface, a recommended parameter change for the patient, wherein the recommended parameter change results in a recommended predicted outcome that differs from the predicted outcome.

8. The method of claim 1, wherein the sensor data is generated by at least one of:
i. a wrist-worn device;
ii. a sweat monitor device;
iii. a blood-sugar monitor device;
iv. a heart monitor device;
v. a pulse oximeter device;
vi. an ear-worn device;
vii. a head-attached device;
viii. an ultrasound wearable device;
ix. an augmented or mixed reality device;
x. an implanted sensor;
xi. a medical device;
xii. a smart contact;
xiii. a smart ring;
xiv. exercise equipment;
xv. a mobile phone;
xvi. a blood-sugar monitor device;
xvii. a respiratory rate monitor device;
xviii. a microphone;
xix. a camera; or
xx. a robotic surgical device.

9. The method of claim 1, wherein the predicted outcome includes at least one of:
a. a predicted functional outcome curve over time;
b. a predicted Patient Report Outcome Measures (PROMs) outcome;
c. a predicted range of motion at a future time;
d. a predicted risk;
e. a predicted cost;
f. a predicted likelihood of suitability of a telehealth follow up for the patient;
g. a predicted discharge location;
h. a predicted likelihood of suitability of virtual physical therapy treatment for the patient;
i. a predicted modifiable risk factor; or
j. a predicted patient score for a procedure, the score being an integer and representing a plurality of outcome predictors.

10. The method of claim 1, wherein the predicted outcome includes two predicted outcomes, including an outcome for a conventional intervention and an outcome for a recommended intervention.

11. The method of claim 1, wherein the at least one medical intervention recommendation includes at least one of a:
a. recommendation for a discharge location;
b. recommendation for a change in a course of care;
c. recommendation of a treatment plan;
d. recommendation for an action to improve the predicted outcome; or
e. recommendation for virtual physical therapy treatment.

12. The method of claim 1, wherein the predicted outcome includes manipulation under anesthesia, wherein the medical intervention undertaken includes breaking up scar tissue, and wherein the updated recommendation includes a less invasive or non-surgical intervention than the at least one medical intervention recommendation.

13. The method of claim 1, wherein the machine learning model is selected based on a surgeon preference from among a plurality of machine learning models, the plurality of machine learning models including at least one of a machine learning model for a high risk patient, a machine learning model for a low risk patient, a traditional machine learning model, a machine learning model based on historical data corresponding to the surgeon, a least invasive recommendation machine learning model, or a patient age based machine learning model, and wherein at least two of the plurality of machine learning models generate different predicted outcomes for the patient.

14. A system comprising:
a data store to store sensor data, the sensor data generated over a period of time by a patient;
a processor;
memory, including instructions, which when executed by the processor, cause the processor to perform operations to:
receive a query to initiate a patient evaluation for the patient;
in response to the query, use the sensor data retrieved from storage as an input to a machine learning model, the machine learning model trained based on labeled sensor data and labeled outcome data and the machine learning model configured to generate a predicted outcome for the patient;
output, for display on a user interface, at least one medical intervention recommendation based on the predicted outcome;
receive updated data related to a medical intervention undertaken by the patient;
determine an updated predicted outcome, using the machine learning model, based on the predicted outcome and the medical intervention; and
output an updated recommendation based on the updated predicted outcome.

15. The system of claim 14, wherein the sensor data is generated by at least two wearable devices of the patient, and further comprising outputting an indication of a data stream of the sensor data most responsible for the predicted outcome.

16. The system of claim 14, wherein the user interface is displayed on a mobile device of the patient that is communicatively coupled to a sensor that generated at least a portion of the sensor data.

17. The system of claim 16, wherein the instructions further include operations to output for display, to the patient on the user interface, a visualization of a surgical intervention corresponding to the at least one medical intervention recommendation, the visualization including at least one of an image, a video, a three-dimensional model, or a four-dimensional model.

18. The system of claim 16, wherein the instructions further include operations to output for display, to the patient on the user interface, an intervention timeline, a recovery period, and an interactive user interface element configured to allow the patient to modify an input parameter to obtain a new predicted outcome based on the modification via the machine learning model.

19. The system of claim 14, wherein the predicted outcome includes manipulation under anesthesia, wherein the medical intervention undertaken includes breaking up scar tissue, and wherein the updated recommendation includes a less invasive or non-surgical intervention than the at least one medical intervention recommendation.

20. The system of claim 14, wherein the machine learning model is selected based on a surgeon preference from among a plurality of machine learning models, the plurality of machine learning models including at least one of a machine learning model for a high risk patient, a machine learning model for a low risk patient, a traditional machine learning model, a machine learning model based on historical data corresponding to the surgeon, a least invasive recommendation machine learning model, or a patient age based machine learning model, and wherein at least two of the plurality of machine learning models generate different predicted outcomes for the patient.
